(19) United States
(12) Patent Application Publication
(10) Pub. No.: US 2006/0121560 A1
(43) Pub. Date: Jun. 8, 2006

(54) DNA fragment to promote translation reaction and method for cell-free protein synthesis system using the same

(75) Inventors: Takashi Suzuki, Osaka (JP); Masaaki Ito, Osaka (JP); Toru Ezure, Osaka (JP); Masamitsu Shikata, Kyoto-shi (JP); Shinichiro Kobayashi, Kyoto-shi (JP)

Correspondence Address: RADER FISHMAN & GRAUER PLLC, LION BUILDING, 1233 20TH STREET N.W., SUITE 501, WASHINGTON, DC, 20036, US

(73) Assignee: SHIMADZU CORPORATION

(21) Appl. No.: 11/295,739

(22) Filed: Dec. 7, 2005

(30) Foreign Application Priority Data
Dec. 7, 2004 (JP) PAT. 2004-354553

(51) Int. Cl. C12P 21/06; C12N 5/06; C12N 15/63

(52) U.S. Cl. 435/68.1; 435/320.1; 435/358

(57) ABSTRACT

The present invention provides a DNA fragment allowing easy cloning of a desired gene and capable of further improving translation efficiency, a protein expression vector and a template DNA having the DNA fragment, a mRNA obtained from the template DNA, a reaction solution for cell-free protein synthesis system containing the template DNA or the mRNA, a method for cell-free protein synthesis system using the template DNA, and, kit for cell-free protein synthesis system including the expression vector. A DNA fragment having the base sequence represented by any of SEQ ID No. 1 to 11 to use for promoting translation reaction, a protein expression vector and a template DNA having the DNA fragment, a mRNA obtained from the template DNA, a reaction solution for cell-free protein synthesis system containing the template DNA or the mRNA, a method for cell-free protein synthesis system using the template DNA, and, kit for cell-free protein synthesis system including the expression vector.

### BACKGROUND OF THE INVENITION

1. Field of the Invention

The present invention relates to a DNA fragment that promotes translation reaction, a protein expression vector and a template DNA having the DNA fragment, a mRNA obtained from the template DNA, a reaction solution for cell-free protein synthesis system containing the template DNA or the mRNA, a method for cell-free protein synthesis system using the template DNA, and, kit for cell-free protein synthesis system including the expression vector.

2. Disclosure of the Related Art

In recent years, genetic information of many organisms, such as human genome, has been decoded. Under the circumstances, functional analysis of proteins and creation of genomic medicine based on such genetic information have been attracting attention for postgenomic studies. Application and utilization of proteins corresponding to such genetic information for pharmaceutical products and the like requires easy synthesis of extensive kinds of proteins in a short time.

At present, expression systems using viable cells (hereinafter sometimes to be referred to as “cell-system”) of yeast, insect cell (insect culture cell) and the like by the gene recombination technique have been widely utilized as the production methods of proteins. However, viable cells show a propensity toward elimination of exogenous proteins for their functional retention, and there are many proteins that cannot be expressed easily since expression of cytotoxic proteins in viable cells prevents cell growth.

On the other hand, as a production method of protein without using viable cell, cell-free protein synthesis system has been known, which includes adding a substrate, an enzyme and the like to a cell rupture, extract solution and the like to provide a wide choice of genetic information translation systems or genetic information transcription/translation systems of organisms in test tubes, and reconstructing a synthetic system capable of linking the necessary number of amino acid residues in a desired order using DNA (transcription template) having a structural gene encoding a target protein or mRNA (translation template). Such a cell-free protein synthesis system is relatively free of the limitation imposed on the above-mentioned cell-system protein synthesis, and is capable of synthesizing proteins without killing the organism. In addition, because the production of protein does not accompany operations of culture and the like, the protein can be synthesized in a short time as compared to cell-systems. Moreover, inasmuch as the cell-free protein synthesis system also affords a large scale production of proteins consisting of amino acid sequences not utilized by the organism, it is expected to be a promising expression method. As an extract solution (extract solution for cell-free protein synthesis system) to be applied to the cell-free protein synthesis system, use of various substances of biological derivation has been considered and investigations are underway.

It is known that eukaryotic mRNA is transcribed from DNA and then undergoes various modifications including splicing, addition of poly-A tail and addition of 5′-cap structure. Additions of poly A tail and 5′-cap structure promote binding of the eukaryotic mRNA to 40s subunit of ribosome. For this reason, conventionally, when an extract solution for cell-free system protein synthesis derived from a eukaryote is used, mRNA capping was conducted by adding a commercially available cap analog to the transcription system in order to achieve efficient translation reaction. However, there was a problem that cap analogs are expensive and significantly reduce the transcription efficiency, and only a small amount of mRNA is obtained. In addition, since unreacted cap analogs inhibit translation reaction, it is necessary to completely remove the unreacted cap analogs after completion of the transcription reaction by means of a spin column or the like. This was a great problem in processing samples with high throughput.

Under such circumstances, Kawarasaki et al. demonstrated that 5′-untranslated region (hereinafter abbreviated as “5′UTR”) derived from tobacco etch virus has a cap-independent translation promoting activity (without forming a cap structure) (see, for example, Kawarasaki et al, “Biotechnol. Prog” Vol. 16, No. 3, p517-521 (2000)). In Kawarasaki et al, “Biotechnol. Prog” Vol. 16, No. 3, p517-521 (2000), there has reported that when mRNA that was transcribed from DNA having 5′UTR derived from tobacco etch virus added upstream side of 5′ of a structural gene encoding a desired protein was used as a template for translation, translation efficiency similar to that of mRNA to which a cap structure was added was realized in cell-free system protein synthesis using a wheat germ extract solution. There has been also reported that in cell-free protein synthesis system using an extract solution derived from rabbit reticulocyte, 5′UTR of rabbit β-globin has a similar function (see, for example, Annweiler et al, “Nucleic acids Res” Vol. 19, No. 13, p3750 (1991)).

As an extract solution for cell-free protein synthesis system, those derived from Escherichia coli, insect culture cell and the like in addition to the aforementioned wheat germ and rabbit reticulocyte are conventionally known. Heretofore, we have proposed cell-free protein synthesis system methods using an extract solution derived from silk worm tissue (silk worm extract solution), an extract solution derived from insect culture cell (insect culture cell extract solution) and an extract solution derived from mammalian culture cell (hereinafter, referred to as mammalian culture cell extract solution) (see, for example, JP-A-2003-235598, JP-A-2004-215651,). These cell-free protein synthesis system methods using the silk worm extract solution, the insect culture cell extract solution and the mammalian culture cell extract solution, that we have proposed, are advantageous because preparation of the extract solution is much easier compared to conventional methods, and synthesis of glycoprotein is enabled due to their eukaryotic origins. Therefore, these methods are very useful. For this reason, finding a cap-independent translation promoting sequence and constructing an expression vector that enables easy cloning of a desired gene are very important challenge in order to rapidly conduct cell-free protein synthesis system with high yield even in cell-free protein synthesis system using such an extract solution.

### SUMMARY OF THE INVENTION

The present invention was devised to solve the aforementioned problems, and it is an object of the present invention to provide a DNA fragment allowing easy cloning of a desired gene and capable of further improving translation efficiency, a protein expression vector and a template DNA having the DNA fragment, a mRNA obtained from the template DNA, a reaction solution for cell-free protein synthesis system containing the template DNA or the mRNA, a method for cell-free protein synthesis system using the template DNA, and, kit for cell-free protein synthesis system including the expression vector.

Through diligent efforts for solving the aforementioned problem, the present inventors finally accomplished the present invention. More specifically, the present invention is as follows.

[1] A DNA fragment of any of the following (a) to (1) used for promoting a translation reaction in a cell-free protein synthesis system:

(a) a DNA fragment having a base sequence represented by SEQ ID No. 1 of the sequence listing;

(b) a DNA fragment having a base sequence represented by SEQ ID No. 2 of the sequence listing;

(c) a DNA fragment having a base sequence represented by SEQ ID No. 3 of the sequence listing;

(d) a DNA fragment having a base sequence represented by SEQ ID No. 4 of the sequence listing;

(e) a DNA fragment having a base sequence represented by SEQ ID No. 5 of the sequence listing;

(f) a DNA fragment having a base sequence represented by SEQ ID No. 6 of the sequence listing;

(g) a DNA fragment having a base sequence represented by SEQ ID No. 7 of the sequence listing;

(h) a DNA fragment having a base sequence represented by SEQ ID No. 8 of the sequence listing;

(i) a DNA fragment having a base sequence represented by SEQ ID No. 9 of the sequence listing;

(j) a DNA fragment having a base sequence represented by SEQ ID No. 10 of the sequence listing;

(k) a DNA fragment having a base sequence represented by SEQ ID No. 11 of the sequence listing; and

(l) a DNA fragment having a base sequence in which one or several base(s) is/are deleted, substituted, inserted or added from/to a base sequence represented by any of SEQ ID Nos. 1-11 of the sequence listing, and having a translation reaction promoting activity.

[2] An expression vector containing at least one DNA fragment selected from the group consisting of the following (a) to (l) having a translation reaction promoting activity:

(a) a DNA fragment having a base sequence represented by SEQ ID No. 1 of the sequence listing;

(b) a DNA fragment having a base sequence represented by SEQ ID No. 2 of the sequence listing;

(c) a DNA fragment having a base sequence represented by SEQ ID No. 3 of the sequence listing;

(d) a DNA fragment having a base sequence represented by SEQ ID No. 4 of the sequence listing;

(e) a DNA fragment having a base sequence represented by SEQ ID No. 5 of the sequence listing;

(f) a DNA fragment having a base sequence represented by SEQ ID No. 6 of the sequence listing;

(g) a DNA fragment having a base sequence represented by SEQ ID No. 7 of the sequence listing;

(h) a DNA fragment having a base sequence represented by SEQ ID No. 8 of the sequence listing;

(i) a DNA fragment having a base sequence represented by SEQ ID No. 9 of the sequence listing;

(j) a DNA fragment having a base sequence represented by SEQ ID No. 10 of the sequence listing; and

(k) a DNA fragment having a base sequence represented by SEQ ID No. 11 of the sequence listing; and

(l) a DNA fragment having a base sequence in which one or several base(s) is/are deleted, substituted, inserted or added from/to a base sequence represented by any of SEQ ID Nos. 1-11 of the sequence listing, and having a translation reaction promoting activity.

[3] A template DNA for cell-free protein synthesis system having a structural gene encoding a protein and a DNA fragment incorporated upstream side of 5′ of the structural gene, wherein the DNA fragment is selected from the group consisting of the following (a) to (l) having a translation reaction promoting activity:

(a) a DNA fragment having a base sequence represented by SEQ ID No. 1 of the sequence listing;

(b) a DNA fragment having a base sequence represented by SEQ ID No. 2 of the sequence listing;

(c) a DNA fragment having a base sequence represented by SEQ ID No. 3 of the sequence listing;

(d) a DNA fragment having a base sequence represented by SEQ ID No. 4 of the sequence listing;

(e) a DNA fragment having a base sequence represented by SEQ ID No. 5 of the sequence listing;

(f) a DNA fragment having a base sequence represented by SEQ ID No. 6 of the sequence listing;

(g) a DNA fragment having a base sequence represented by SEQ ID No. 7 of the sequence listing;

(h) a DNA fragment having a base sequence represented by SEQ ID No. 8 of the sequence listing;

(i) a DNA fragment having a base sequence represented by SEQ ID No. 9 of the sequence listing;

(j) a DNA fragment having a base sequence represented by SEQ ID No. 10 of the sequence listing; and

(k) a DNA fragment having a base sequence represented by SEQ ID No. 11 of the sequence listing; and

(l) a DNA fragment having a base sequence in which one or several base(s) is/are deleted, substituted, inserted or added from/to a base sequence represented by any of SEQ ID Nos. 1-11 of the sequence listing, and having a translation reaction promoting activity.

[4] A mRNA obtained by transcription from the template DNA according to [3] and used as a transcription template in cell-free protein synthesis system.

[5] A reaction solution for cell-free protein synthesis system including the template DNA according to [3] or the mRNA obtained by transcription from the template DNA.

In the present invention, the “solution” encompasses the suspension.

[6] A method for cell-free protein synthesis system using the template DNA according to [3] or the mRNA obtained by transcription from the template DNA.

[7] The method for cell-free protein synthesis system according to [6], using a reaction solution for cell-free protein synthesis system including an animal-derived extract.

[8] The method for cell-free protein synthesis system according to [7], wherein the animal-derived extract is extracted from a silk worm tissue.

[9] The method for cell-free protein synthesis system according to [7], wherein the animal-derived extract is extracted from an insect culture cell.

[10] The method for cell-free protein synthesis system according to [9], wherein the insect culture cell is a cell derived from Trichoplusia ni egg cell and/or Spodoptera frugiperda ovary cell.

[11] The method for cell-free protein synthesis system according to [7], wherein the animal-derived extract is extracted from a mammalian cell.

[12] The method for cell-free protein synthesis system according to [11], wherein the mammalian cell is a rabbit reticulocyte.

[13] The method for cell-free protein synthesis system according to [11], wherein the mammalian cell is a mammalian culture cell.

[14] The method for cell-free protein synthesis system according to [13], wherein the mammalian culture cell is a Chinese hamster ovary cell.

[15] The method for cell-free protein synthesis system according to [6], using a reaction solution for cell-free protein synthesis system including a wheat germ extract.

[16] A kit for cell-free protein synthesis system including the expression vector according to [2].

According to the protein expression vector of the present invention, it is possible to readily conduct cloning of a desired gene, and to further improve the translation efficiency.

### BRIEF DESCRIPTION OF THE DRAWINGS

FIG. 1 is a graph showing a result of Experiment Example 2.

FIG. 2 is a graph showing a result of Experiment Example 3.

FIG. 3 is a graph showing a result of Experiment Example 4.

FIG. 4 is a graph showing a result of Experiment Example 5.

FIG. 5 is a graph showing a result of Example 1.

FIG. 6 is a graph showing a result of Example 2.

FIG. 7 is a vector map of expression vector pTD1 produced in Reference Example 24.

FIG. 8 is a vector map of expression vector pTD2 produced in Reference Example 25.

FIG. 9 is a graph showing a result of Example 3.

FIG. 10 is a graph showing a result of Example 4.

### DETAILED DESCRIPTION OF THE INVENTION

In the following, the present invention will be explained in more detail.

<DNA Fragment>

A DNA fragment of the present invention has a translation reaction promoting activity, without depending on the cap structure, in a protein expression system. The phrase “having a translation reaction promoting activity” used herein means that a synthesis amount of protein is improved (for example 1.2 times or more, preferably 2 times or more) by conducting cell-free protein synthesis system reaction using the DNA fragment of the present invention in comparison with the case where the DNA fragment is not used.

Although a cell-free protein synthesis system is used as means for detecting easily an effect of the DNA fragment that promotes a translation reaction, the expression vector of the present invention may be used in a conventionally known cell system without limitation to the cell-free system.

Concrete examples of the DNA fragment having a translation reaction promoting activity and being non-dependent on the cap structure of the present invention include double-stranded DNA fragments having a base sequence represented by any of SEQ ID Nos. 1-11 of the sequence listing. These DNA fragments also include double-stranded DNA fragments having equivalent base sequences (one or several base(s) is/are deleted, substituted, inserted or added from/to the base sequences represented by any of SEQ ID Nos. 1-11 of the sequence listing) and having a translation reaction promoting activity.

Base sequences represented by any of SEQ ID Nos. 1-11 of the sequence listing are respectively base sequences known as 5′-untranslated regions (5′UTR) in silk worm and baculovirus. To be more specific,

(1-1) the base sequence represented by SEQ ID No. 1 is known as a base sequence of 5′UTR of fibroin L-chain gene of silk worm;

(1-2) the base sequence represented by SEQ ID No. 2 is known as a base sequence of 5′UTR of sericin gene of silk worm;

(1-3) the base sequence represented by SEQ ID No. 3 is known as a base sequence of 5′UTR of polyhedrin gene of AcNPV (Autographa californica nuclear polyhedrosis virus);

(1-4) the base sequence represented by SEQ ID No. 4 is known as a base sequence of 5′UTR of polyhedrin gene of BmCPV (Bombyx mori cytoplasmic polyhedrosis virus);

(1-5) the base sequence represented by SEQ ID No. 5 is known as a base sequence of 5′UTR of polyhedrin gene of EsCPV (Euxoa scandes cytoplasmic polyhedrosis virus);

(1-6) the base sequence represented by SEQ ID No. 6 is known as a base sequence of 5′UTR of polyhedrin gene of HcNPV (Hyphantria cunea nuclear polyhedrosis virus);

(1-7) the base sequence represented by SEQ ID No. 7 is known as a base sequence of 5′UTR of polyhedrin gene of CrNPV (Choristoneura rosaceana nucleopolyhedrovirus);

(1-8) the base sequence represented by SEQ ID No. 8 is known as a base sequence of 5′UTR of polyhedrin gene of EoNPV (Ecotropis oblique nuclear polyhedrosis virus);

(1-9) the base sequence represented by SEQ ID No. 9 is known as a base sequence of 5′UTR of polyhedrin gene of MnNPV (Malacosma neustria nuclecopolyhedrovirus);

(1-10) the base sequence represented by SEQ ID No. 10 is known as a base sequence of 5′UTR of polyhedrin gene of SfNPV (Spodoptera frugiperda nucleopolyhedrovirus); and

(1-11) the base sequence represented by SEQ ID No. 11 is known as a base sequence of 5′UTR of polyhedrin gene of WsNPV (Wiseana signata nucleopolyhedrovirus).

The present invention found that DNA fragments having these base sequences and equivalent DNA fragment not missing the functionality exert especially useful translation reaction promoting activity in the cell-free system protein synthesis system. As long as the DNA fragments of the present invention may be derived from 5′UTR of silk worm or baculovirus, they need not necessarily have the aforementioned base sequences.

The DNA fragments of the present invention may be obtained in any known methods. For example, they may be synthesized by a known DNA synthesizer.

<Expression Vector>

Preferably, one or a plurality of the DNA fragment(s) of the present invention is/are incorporated upstream side of 5′ of the structural gene encoding protein, to be constructed as an expression vector. The vector is also comprised in the present invention. The vector of the present invention may be chain or cyclic.

Among the vectors of the present invention, those exert significant translation reaction promoting activity and thus are especially preferred will be exemplified below.

(2-1) An expression vector with one DNA fragment comprising the base sequence represented by SEQ ID No. 2 or one DNA fragment comprising a base sequence equivalent thereto and having a translation reaction promoting activity, incorporated downtsream side of 3′ of a promoter sequence in forward direction;

(2-2) An expression vector with one DNA fragment comprising the base sequence represented by SEQ ID No. 3 or one DNA fragment comprising a base sequence equivalent thereto and having a translation reaction promoting activity, incorporated downtsream side of 3′ of a promoter sequence in forward direction;

(2-3) An expression vector with one DNA fragment comprising the base sequence represented by SEQ ID No. 4 or one DNA fragment comprising a base sequence equivalent thereto and having a translation reaction promoting activity, incorporated downtsream side of 3′ of a promoter sequence in forward direction;

(2-4) An expression vector with two DNA fragments, which have the same or different base sequences and are selected from the group consisting of DNA fragment comprising the base sequence represented by SEQ ID No. 5 and DNA fragment comprising a base sequence equivalent thereto and having a translation reaction promoting activity, said two DNA fragments being incorporated downtsream side of 3′ of a promoter sequence in reverse direction;

(2-5) An expression vector with one DNA fragment comprising the base sequence represented by SEQ ID No. 6 or one DNA fragment comprising a base sequence equivalent thereto and having a translation reaction promoting activity, incorporated downtsream side of 3′ of a promoter sequence in reverse direction;

(2-6) An expression vector with two DNA fragments, which have the same or different base sequences and are selected from the group consisting of DNA fragment comprising the base sequence represented by SEQ ID No. 6 and DNA fragment comprising a base sequence equivalent thereto and having a translation reaction promoting activity, said two DNA fragments being incorporated downtsream side of 3′ of a promoter sequence in reverse direction;

(2-7) An expression vector with one DNA fragment comprising the base sequence represented by SEQ ID No. 7 or one DNA fragment comprising a base sequence equivalent thereto and having a translation reaction promoting activity, incorporated downtsream side of 3′ of a promoter sequence in forward direction;

(2-8) An expression vector with one DNA fragment comprising the base sequence represented by SEQ ID No. 7 or one DNA fragment comprising a base sequence equivalent thereto and having a translation reaction promoting activity, incorporated downtsream side of 3′ of a promoter sequence in reverse direction;

(2-9) An expression vector with one DNA fragment comprising the base sequence represented by SEQ ID No. 8 or one DNA fragment comprising a base sequence equivalent thereto and having a translation reaction promoting activity, incorporated downtsream side of 3′ of a promoter sequence in reverse direction;

(2-10) An expression vector:

with one DNA fragment which is selected from the group consisting of DNA fragment comprising the base sequence represented by SEQ ID No. 9 and DNA fragment comprising a base sequence equivalent thereto and having a translation reaction promoting activity, or

with two DNA fragments which have the same or different sequences and are selected from said group,

said DNA fragment (s) being incorporated downtsream side of 3′ of a promoter sequence in forward direction;

(2-11) An expression vector with one DNA fragment comprising the base sequence represented by SEQ ID No. 9 or one DNA fragment comprising a base sequence equivalent thereto and having a translation reaction promoting activity, incorporated downtsream side of 3′ of a promoter sequence in reverse direction;

(2-12) An expression vector with one DNA fragment comprising the base sequence represented by SEQ ID No. 10 or one DNA fragment comprising a base sequence equivalent thereto and having a translation reaction promoting activity, incorporated downtsream side of 3′ of a promoter sequence in forward direction; and

(2-13) An expression vector with one DNA fragment comprising the base sequence represented by SEQ ID No. 11 or one DNA fragment comprising a base sequence equivalent thereto and having a translation reaction promoting activity, incorporated downtsream side of 3′ of a promoter sequence in forward direction.

The expression vector of the present invention usually has at least one promoter sequence upstream side of 5′ of the aforementioned DNA fragments. Examples of the promoter sequence include conventionally known T7 promoter sequence, SP6 promoter sequence, T3 promoter sequence, and the like.

The expression vector of the present invention contains one or a plurality of the aforementioned DNA fragments. The DNA fragments may be incorporated in forward direction (5′→3′) downstream side of 3′ of a promoter sequence, or may be incorporated in reverse direction. When the plurality of DNA fragments are included, the DNA fragments may be the same as or different from each other. When two or more DNA fragments are incorporated, it is not necessary that all of the DNA fragments are incorporated in the same direction.

The expression vector of the present invention has a sequence for allowing insertion of a structural gene encoding a protein to be expressed. Examples of the sequence for allowing insertion include conventionally known multi-cloning site, a sequence causing homologous recombination reaction, and the like. Such a sequence for allowing insertion of a structural gene encoding a protein is incorporated downstream side of 3′ of the DNA fragment having the translation reaction promoting activity. From the viewpoint of facilitating purification of the expressed protein, a base sequence such that encodes conventionally known histidine tag or GST tag may be added to the sequence for allowing insertion of a structural gene.

Preferably, the expression vector of the present invention has a 3′-untranslated region (3′UTR) and a poly-A sequence downstream side of 3′ of the sequence for allowing insertion of a structural gene encoding a protein, from the viewpoint of stability of synthesized mRNA and the like.

Preferably, the expression vector of the present invention has a terminator sequence having a function of terminating transcription downstream side of 3′ of the poly-A sequence. Examples of the terminator sequence include conventionally known T7 terminator sequence, SP6 terminator sequence, T3 terminator sequence, and the like.

The expression vector of the present invention has a drug resistance marker so as to be stably retained in a host. Examples of the drug resistance marker include conventionally known ampicillin resistant gene, kanamycin resistant gene, and the like.

The expression vector of the present invention has an origin of replication for enabling autonomous replication in a host. Examples of the origin of replication include conventionally known pBR322 Ori, pUC Ori, SV40 Ori, and the like. It may have an origin of replication that functions in different hosts so as to allow use as a shuttle vector.

These expression vectors may be created by using conventionally known gene recombination techniques.

To the above-described expression vector of the present invention, a structural gene encoding a target protein (including peptide) to be synthesized in a cell-free system is inserted. There is no specific restriction for the protein (including peptide) encoded by the structural gene, and the structure gene may have a base sequence encoding a protein which turns to be cytotoxic in a living cell, or may have a base sequence encoding a glycoprotein, or may be a base sequence encoding a fusion protein. From the viewpoint of facilitating purification of the expressed protein, a base sequence such that encodes conventionally known histidine tag or GST tag may be added. These tag sequences are usually added to an N terminal or C terminal of the target protein.

As to the structural gene, there is no specific restriction for its number of bases, and every gene does not necessarily have the same number of bases insofar as the target protein can be synthesized. Each structural gene may have deletion, substitution, insertion and addition of a plurality of bases insofar as it has such a homogenous sequence that allows synthesis of the target protein.

<Template DNA>

A vector in which a structural gene encoding a target protein (including peptide) is inserted into the expression vector of the present invention (hereinafter, referred to as template DNA) may be used in a cell system and a cell-free system. Namely, it is also preferable that one or a plurality of the DNA fragment(s) of the present invention is incorporated upstream side of 5′ of the structural gene encoding protein, to be constructed as a template DNA. The template DNA is also comprised in the present invention. The template DNA may be chain or cyclic. In the template DNA, the DNA fragment may be incorporated in forward direction (5′→3′) upstream side of 5′ of the structural gene, or may be incorporated in reverse direction (3′→5′). Further, in the template DNA of the present invention, two or more DNA fragments may be incorporated, and in this case, the incorporated DNA fragments may be the same as or different from each other. When two or more DNA fragments are incorporated, it is not necessary that all of the DNA fragments are incorporated in the same direction. The DNA fragment may be incorporated upstream side of 5′ of the structural gene so as to adjoin the structural gene, or to allow a base sequence having one or more base(s) to intervene between the DNA fragment and the structural gene. The template DNA may be appropriately constructed by applying the known gene manipulation technique.

The structural gene in the template DNA of the present invention is a region encoding the target protein to be synthesized in cell-free system. There is no specific restriction for the protein (including peptide) encoded by the structural gene, and the structure gene may have a base sequence encoding a protein which turns to be cytotoxic in a living cell, or may have a base sequence encoding a glycoprotein, or may be a base sequence encoding a fusion protein.

The template DNA of the present invention usually has at least one promoter sequence upstream side of 5′ of the aforementioned DNA fragments. Examples of the promoter sequence include conventionally known T7 promoter sequence, SP6 promoter sequence, T3 promoter sequence, and the like.

Preferably, the template DNA of the present invention also has a terminator sequence having a function of terminating transcription, and/or a poly-A sequence from the viewpoint of stability of synthesized mRNA and the like, downstream side of 3′ of the structural gene. Examples of the terminator sequence include conventionally known T7 terminator sequence, SP6 terminator sequence, T3 terminator sequence, and the like.

Among the template DNA of the present invention, those exert significant translation reaction promoting activity and thus are especially preferred will be exemplified below.

(3-1) A template DNA with one DNA fragment comprising the base sequence represented by SEQ ID No. 2 or one DNA fragment comprising a base sequence equivalent thereto and having a translation reaction promoting activity, incorporated upstream side of 5′ of a structural gene in forward direction;

(3-2) A template DNA with one DNA fragment comprising the base sequence represented by SEQ ID No. 3 or one DNA fragment comprising a base sequence equivalent thereto and having a translation reaction promoting activity, incorporated upstream side of 5′ of a structural gene in forward direction;

(3-3) A template DNA with one DNA fragment comprising the base sequence represented by SEQ ID No. 4 or one DNA fragment comprising a base sequence equivalent thereto and having a translation reaction promoting activity, incorporated upstream side of 5′ of a structural gene in forward direction;

(3-4) A template DNA with two DNA fragments, which have the same or different base sequences and are selected from the group consisting of DNA fragment comprising the base sequence represented by SEQ ID No. 5 and DNA fragment comprising a base sequence equivalent thereto and having a translation reaction promoting activity, said two DNA fragments being incorporated upstream side of 5′ of a structural gene in reverse direction;

(3-5) A template DNA with one DNA fragment comprising the base sequence represented by SEQ ID No. 6 or one DNA fragment comprising a base sequence equivalent thereto and having a translation reaction promoting activity, incorporated upstream side of 5′ of a structural gene in reverse direction;

(3-6) A template DNA with two DNA fragments, which have the same or different base sequence and are selected from the group consisting of DNA fragment comprising the base sequence represented by SEQ ID No. 6 and DNA fragment comprising a base sequence equivalent thereto and having a translation reaction promoting activity, said two DNA fragments being incorporated upstream side of 5′ of a structural gene in reverse direction;

(3-7) A template DNA with one DNA fragment comprising the base sequence represented by SEQ ID No. 7 or one DNA fragment comprising a base sequence equivalent thereto and having a translation reaction promoting activity, incorporated upstream side of 5′ of a structural gene in forward direction;

(3-8) A template DNA with one DNA fragment comprising the base sequence represented by SEQ ID No. 7 or one DNA fragment comprising a base sequence equivalent thereto and having a translation reaction promoting activity, incorporated upstream side of 5′ of a structural gene in reverse direction;

(3-9) A template DNA with one DNA fragment comprising the base sequence represented by SEQ ID No. 8 or one DNA fragment comprising a base sequence equivalent thereto and having a translation reaction promoting activity, incorporated upstream side of 5′ of a structural gene in reverse direction;

(3-10) A template DNA with two DNA fragments, which have the same or different base sequence and are selected from the group consisting of DNA fragment comprising the base sequence represented by SEQ ID No. 9 and DNA fragment comprising a base sequence equivalent thereto and having a translation reaction promoting activity, said two DNA fragments being incorporated upstream side of 5′ of a structural gene in forward direction;

(3-11) A template DNA with one DNA fragment comprising the base sequence represented by SEQ ID No. 9 or one DNA fragment comprising a base sequence equivalent thereto and having a translation reaction promoting activity, incorporated upstream side of 5′ of a structural gene in reverse direction;

(3-12) A template DNA with one DNA fragment comprising the base sequence represented by SEQ ID No. 10 or one DNA fragment comprising a base sequence equivalent thereto and having a translation reaction promoting activity, incorporated upstream side of 5′ of a structural gene in forward direction; and

(3-13) A template DNA with one DNA fragment comprising the base sequence represented by SEQ ID No. 11 or one DNA fragment comprising a base sequence equivalent thereto and having a translation reaction promoting activity, incorporated upstream side of 5′ of a structural gene in forward direction.

The aforementioned template DNA may be preferably used as a transcription template in cell-free protein synthesis system. Namely, in general, cell-free protein synthesis system can be broadly classified into protein synthesis (translation system) based only on the cell-free translation system in which a protein is synthesized from read information of mRNA (translation template), as well as protein synthesis (transcription/translation system) comprising a transcription step in which mRNA is transcribed from DNA (transcription template) and a translation step in which a protein is synthesized by reading information of mRNA obtained in the transcription step. Among these, the template DNA of the present invention may be preferably used as a transcription template in cell-free protein synthesis system by transcription/translation system.

<mRNA>

The mRNA obtained by transcription from the template DNA of the present invention may be preferably used as a translation template in cell-free protein synthesis system by translation system. The mRNA obtained by transcription from the template DNA is also comprised in the scope of the present invention. The mRNA of the present invention may be prepared by transcription from the template DNA according appropriately to the conventionally known technique, and preferably by transcription from the template DNA by in vitro transcription which itself is known. In vitro transcription may be performed by using, for example, RiboMax Large Scale RNA production System-T7 (manufactured by Promega Corporation) and the like. After transcription, mRNA is purified by the method which itself is known to be isolated, and may be applied to a reaction solution for translation system as a translation template as described after.

When a template DNA is used in a cell system, the template DNA is introduced into a host organism in a conventionally known manner to obtain a transformant. Any living species may be used as the host used in this case. In particular, since a DNA fragment having a translation reaction promoting activity contained in the expression vector is derived from silk worm or baculovirus, using in a baculovirus expression system or a cell system using silk worm is especially preferred.

<Reaction Solution for Cell-Free Protein Synthesis System and Method for Cell-Free Protein Synthesis System>

In general, cell-free protein synthesis system can be broadly classified into protein synthesis based only on the cell-free translation system in which a protein is synthesized from read information of mRNA (translation system) and protein synthesis comprising a transcription step in which mRNA is transcribed from DNA and a translation step in which a protein is synthesized by reading information of mRNA obtained in the transcription step (transcription/translation system). The template DNA may be preferably used in either system. Namely, the template DNA or the mRNA obtained by transcription from the template DNA is used as a reaction template. In the present invention, a method for cell-free protein synthesis system using the template DNA or the mRNA obtained by transcription from the template DNA is comprised.

In the present invention, a reaction solution for cell-free protein synthesis system using the template DNA or the mRNA obtained by transcription from the template DNA as a reaction template is also comprised. The reaction solution for cell-free protein synthesis system is preferably used for the method for cell-free protein synthesis system of the present invention. The reaction solution for cell-free protein synthesis system of the present invention may be any formation of a reaction solution for conducting synthesis reaction by translation system (hereinafter referred as “a reaction solution for translation system”) and a reaction solution for synthesis reaction by transcription/translation system (hereinafter referred as “a reaction solution for transcription/translation system”). Namely, the reaction solution for cell-free protein synthesis system may be the reaction solution for translation system including the template DNA as a transcription template, and may be the reaction solution for transcription/translation system including the mRNA obtained by transcription from the template DNA as a translation template.

The reaction solution for cell-free protein synthesis system usually includes living body-derived extract including ribosome as a translation device and the like. Further, the extract in the reaction solution for cell-free protein synthesis system of the present invention may be any extract solution insofar as it allows generation of the protein encoded by the template DNA, and extracts and extract solutions extracted from conventionally known Escherichia coli, gramineous plants such as wheat, barley, rice and corn, germ of vegetable seed such as spinach, rabbit reticulocyte, and the like may be used without any particular restriction. These may be commercially available ones, or may be prepared in accordance with a per se well-known method, concretely like a method as described in Zubay G “Ann Rev Genet” Vol. 7, p267-287 (1973) in the case of Escherichia coli extract solution. Examples of the commercially available cell extract solution for protein synthesis include E. coli S30 extract for linear templates (manufactured by Promega Corporation) and the like when the extract solution is derived from E. coli, rabbit reticulocyte lysate systems (manufactured by Promega Corporation) and the like when the extract solution is derived from rabbit reticulocyte, wheat germ extract (manufactured by Promega Corporation), PROTEIOS (manufactured by TOYOBO Co., Ltd.) derived from wheat germ, and the like when the extract solution is derived from wheat germ.

In a reaction solution for cell-free protein synthesis system may include the known extracts or extract solutions as described above, however, it is preferred that an extract derived from animal included as has been proposed by the present inventors. Examples of such an extract derived from animal include extracts derived from arthropod, extracts derived from mammalian culture cell, and the like.

The extract solution derived from arthropod may be extracted from any tissues regardless of the growth stage of the arthropod, and it may be extracted from culture cell derived from any tissues of the arthropod. In particular, those extracted from silk worm tissue or insect culture cell are preferably used. When silk worm tissue is used for extraction, inclusion of an extract from posterior silk gland of young silk worm at 3 to 7 days in the fifth period is particularly preferred because a reaction solution for cell-free protein synthesis system capable of synthesizing a large amount of proteins in a short time is advantageously obtained (see JP-A-2003-235598). When insect culture cell is used for extraction, a cell High Five (manufactured by Invitrogen Corporation) derived from egg cell of Trichoplusia ni and a cell Sf21 (manufactured by Invitrogen Corporation) derived from ovary cell of Spodoptera frugiperda which can exhibit high protein synthesis ability and can be cultured in a serum free medium may be exemplified as a preferred insect culture cell (see JP-A-2004-215651).

As an extract solution derived from the mammalian culture cell, conventionally known culture cells derived from mammalians such as human, rat, mouse and monkey may be appropriately used without any specific limitation.

As the mammalian culture cell, cells derived from any tissues may be used, for example, blood cells, testis-derived cells, lymphoma-derived cells, and other tumor cells, stem cells, and the like may be used without any specific limitation. In particular, lymphoma-derived cells are preferably used because they are culturable in suspension culture and hence easy to be cultivated and subcultured. Moreover, Chinese hamster ovary (CHO) K1-SFM cells are not only culturable in suspension culture but also culturable in a serum-free medium, so that they are easier to be cultured and subcultured. Additionally, CHO K1-SFM cells are widely used in cell systems and have high ability to synthesize proteins, and similar features are expected to be exerted also in cell-free systems. Therefore, use of CHO K1-SFM cells is preferred.

Not limited to mammalian culture cells derived from a single kind of tissue in a single species of mammalian, extraction may be conducted from mammalian culture cells derived from plural kinds of tissues in a single species of mammalian, or extraction may be conducted from mammalian culture cells derived from a single kind of tissue in plural species of mammalian. Of course, extraction may be conducted from mammalian culture cells derived from plural kinds of tissues in plural species of mammalian.

A solution for extraction to be used in the extraction operation from the tissue or culture cell derived from the animal is not particularly limited, but it preferably contains at least a protease inhibitor. When a solution for extraction containing a protease inhibitor is used, the protease activity contained in an extract is inhibited, thereby preventing undesired decomposition of the active protein in the extract due to protease, which in turn effectively draws out advantageously the protein synthesis ability that the extract derived from the cultured mammalian cell has. The above-mentioned protease inhibitor is not particularly limited as long as it can inhibit the activity of protease, and, for example, phenylmethanesulfonyl fluoride (hereinafter sometimes to be referred to as “PMSF”), aprotinin, bestatin, leupeptin, pepstatin A, E-64 (L-trans-epoxysuccinyl-L-leucylamido(4-guanidino)butane), ethylenediaminetetraacetic acid, phosphoramidon and the like can be used. Since an extract often contains serine protease, the use of PMSF, which works as an inhibitor having high specificity to serine protease, is preferable among those mentioned above. It is possible to use not only one kind of protease inhibitor but also a mixture (protease inhibitor cocktail) of several kinds of protease inhibitors.

The content of the protease inhibitor in the solution for extraction is free of any particular limitation, but it is preferably 1 μM-50 mM, more preferably 0.01 mM-5 mM, because decomposition of the enzyme necessary for the action of the present invention can be preferably inhibited. This is because the decomposition activity of protease often cannot be suppressed sufficiently when the protease inhibitor content is less than 1 μM, and the protein synthesis reaction tends to be inhibited when the protease inhibitor content exceeds 50 mM.

The solution for extraction to be used for the tissue or the culture cell derived from the animal preferably contains, in addition to the above-mentioned protease inhibitor, at least a potassium salt, a magnesium salt, dithiothreitol and a buffer.

The above-mentioned potassium salt may be used in a general form, such as potassium acetate, potassium carbonate, potassium hydrogen carbonate, potassium chloride, dipotassium hydrogen phosphate, dipotassium hydrogen citrate, potassium sulfate, potassium dihydrogen phosphate, potassium iodide, potassium phthalate and the like, with preference given to potassium acetate. Potassium salt acts as a cofactor in the protein synthesis reaction.

The content of the potassium salt in the solution for extraction is free of any particular limitation, but from the aspect of preservation stability, it is preferably 10 mM-500 mM, more preferably 20 mM-300 mM, in the case of a monovalent potassium salt, such as potassium acetate and the like. When the content of the potassium salt is less than 10 mM or more than 500 mM, the components essential for protein synthesis tend to become unstable.

The above-mentioned magnesium salt may be used in a general form such as magnesium acetate, magnesium sulfate, magnesium chloride, magnesium citrate, magnesium hydrogen phosphate, magnesium iodide, magnesium lactate, magnesium nitrate, magnesium oxalate and the like, with preference given to magnesium acetate. Magnesium salt also acts as a cofactor in the protein synthesis reaction.

The content of the magnesium salt in the solution for extraction is free of any particular limitation, but from the aspect of preservation stability, it is preferably 0.1 mM-10 mM, more preferably 0.5 mM-5 mM, in the case of a divalent salt, such as magnesium acetate and the like. When the content of the magnesium salt is less than 0.1 mM or more than 10 mM, the components essential for protein synthesis tend to become unstable.

The above-mentioned DTT is added for prevention of oxidization, and is preferably contained in an amount of 0.1 mM-10 mM, more preferably 0.5 mM-5 mM, in the solution for extraction. When the content of DTT is less than 0.1 mM or more than 10 mM, the components essential for protein synthesis tend to become unstable.

The above-mentioned buffer imparts a buffer capacity, and is, added for prevention of denaturation of the extract caused by a radical change in pH of the extract solution, which is due to, for example, addition of an acidic or basic substance and the like. Such buffer is free of any particular limitation, and, for example, HEPES-KOH, Tris-HCl, acetic acid-sodium acetate, citric acid-sodium citrate, phosphoric acid, boric acid, MES, PIPES and the like may be used.

The buffer is preferably one that maintains the pH of the obtained extract solution at 4-10, more preferably pH 6.0-8.5. When the pH of the extract solution is less than 4 or more than 10, the components essential for the reaction of the present invention may be denatured. From this aspect, the use of HEPES-KOH (pH 6.0-8.5) is particularly preferable among the above-mentioned buffers.

While the content of the buffer in the solution for extraction is free of any particular limitation, it is preferably 5 mM-200 mM, more preferably 10 mM-100 mM, to maintain preferable buffer capacity. When the content of the buffer is less than 5 mM, pH tends to change radically due to the addition of an acidic or basic substance, which in turn may cause denaturation of the extract in the extract solution prepared using less than 5 mM of the buffer, and when the content of the buffer exceeds 200 mM, the salt concentration becomes too high and the components essential for protein synthesis tend to become unstable.

Further, in the case that the object for the extraction is culture cell of arthropod or mammalian animal, in order to improve the capacity for protein synthesis of the obtained extract solution, preferably calcium salt and glycerol are further added. The calcium salt is not particularly limited and may be used in a general form, such as calcium chloride, calcium acetate, calcium sulfate, calcium citrate, calcium iodide, calcium lactate, calcium nitrate, calcium oxalate and the like, with preference given to calcium chloride. In this case, the content of calcium chloride is not particularly limited. For effective exertion of the effect of the above-mentioned improved protein synthesis ability, it is preferably contained in the range of 0.1 mM-10 mM, more preferably 0.5 mM-5 mM. In addition, while the amount of glycerol to be added is not particularly limited, for effective exertion of the effect of the above-mentioned improved protein synthesis ability, it is preferably added in a proportion of (v/v)%-80 (v/v)%, more preferably 10 (v/v)%-50 (v/v)%.

The aforementioned extract solutions derived from arthropod may be obtained by appropriately conducting a conventionally known extraction operation, however, it is preferable that extract solutions derived from silk worm tissue are prepared by the method described in JP-A-2003-235598 and extract solutions derived from culture cell are prepared by the method described in JP-A-2004-215651 since particularly high activity of protein synthesis is realized with simple extraction operations.

Also the method of crushing cells in preparation of an extract derived from mammalian culture cell is not particularly limited, and conventionally known method may be appropriately used. In particular, a method of crushing cells by freezing and thawing is preferred. Since the above method allows crushing of cells in a gentler condition compared to the conventional method, and components essential for protein synthesis can be taken out without being broken, it is possible to readily prepare a mammalian culture cell extract realizing higher amount of protein synthesis than the conventional one in a cell-free system.

In the cell crushing method of mammalian culture cell, it is necessary to rapidly freeze mammalian culture cells suspended in a solution for extraction. In such a crushing method, “rapidly freeze” means freezing mammalian culture cells in not more than 10 seconds, preferably not more than 2 seconds. If freezing of the mammalian culture cells is not conducted rapidly, components essential for protein synthesis may be in activated, so that the aforementioned effect of the extraction method is not achieved.

As described above, the temperature at which the mammalian culture cells are rapidly frozen is usually not more than −80° C., and preferably not more than −150° C. If the cells are rapidly frozen at a temperature exceeding −80° C., components essential for protein synthesis are inactivated and the ability of protein synthesis tends to decrease.

The rapid freezing of mammalian culture cells may be realized by using inert gas such as liquid nitrogen or liquid helium, however, it is preferred to use liquid nitrogen because it is cheap and readily available.

In centrifugally separating the above rapidly frozen mammalian culture cells after thawing, thawing may be realized by thawing in a water bath or ice water bath at, for example, −10° C. to 20° C., or leaving at room temperature (25° C.). In order to prevent components essential for protein synthesis from being inactivated and to securely prevent deterioration of protein synthesis ability, thawing is preferably conducted in a water bath or ice water bath at 0° C. to 20° C. (in particular, 4° C. to 10° C.).

Centrifugal separation of the thawed mammalian culture cells may be conducted in the condition usually employed in the art (10,000×g-50,000×g, 0° C.-10° C., 10 minutes-60 minutes)

In the preparation method of a mammalian culture cell extract, procedures following crushing of cells till obtaining a mammalian culture cell extract for cell-free protein synthesis system are not particularly limited.

For example, when thawing and centrifugation are conducted after the step of rapidly freezing the mammalian culture cells suspended in a solution for extraction, the supernatant (supernatant 1) obtained by this centrifugation may be directly used as a mammalian culture cell extract solution, or the supernatant 1 may further be centrifuged and the resultant supernatant (supernatant 2) may be used as a mammalian culture cell extract solution. Centrifugation of the supernatant 1 may be conducted in the same condition as described above (10,000×g-50,000×g, 0° C.-10° C., 10 minutes-60 minutes).

After preparing extracts as described above, gel filtration may be conducted, and fractions with absorbance at 280 nm of 10 or more may be collected from a filtered solution after gel filtration to prepare as an extract solution.

Preferably, mammalian culture cells subjected to a preparation method are washed in advance with a washing solution prior to rapid freezing for preventing a medium used for culture from entering the translation reaction solution. Compositions of the washing solution may be those of the solution for extraction as described above. Washing with the washing solution is conducted by adding the washing solution to the mammalian culture cells and centrifuging the resultant solution (for example, in the condition of 700×g, 10 minutes, 4° C.).

An amount of the washing solution used for the washing is preferably 5 mL-100 mL, more preferably 10 mL-50 mL relative to 1 g in wet weight of mammalian culture cells, from the viewpoint of completely washing out the culture medium.

The number of times of washing is preferably 1-5, more preferably 2-4.

The amount of mammalian culture cell is not particularly limited, but is preferably 0.1 g-5 g, more preferably 0.5 g-2 g relative to 1 mL of the solution for extraction in order to keep the optimum extraction efficiency.

The content of the extract included in the extract solution derived from mammalian culture cells is not particularly limited, however, it is preferably 1 mg/mL-200 mg/mL in terms of protein concentration, more preferably 10 mg/mL-100 mg/mL. If the content of the extract is less than 1 g/mL in terms of protein concentration, concentration of components essential for cell-free protein synthesis system is low, so that sufficient synthesis reaction is unlikely to be achieved. If the content of the extract exceeds 200 mg/mL in terms of protein concentration, the extract solution is likely to have high viscosity to make it difficult to operate.

The extract solution containing the amount within the above range of the extract may be prepared using protein concentration measurement of the extract solution. The protein concentration measurement may be conducted in a procedure usually employed in the art such that 0.1 mL of sample is added to 2 mL a reaction reagent using, for example, BCA Protein assay Kit (manufactured by PIERCE BIOTECHNOLOGY, Inc.) and allowed to react at 37° C. for 30 minutes, and absorbance at 562 nm is measured. Using a spectrometer (Ultrospec3300pro, manufactured by Amersham Biosciences), absorbance at 562 nm is measured. As a control, bovine serum albumin (BSA) is usually used.

Preferably, an extract solution derived from mammalian culture cell is realized such that it contains 10 mg/mL-100 mg/mL of extract in terms of protein concentration, 20 mM-300 mM of potassium acetate, 0.5 mM-5 mM of magnesium acetate, 0.5 mM-5 mM of DTT, 0.01 mM-5 mM of PMSF, and 10 mM-100 mM of HEPES-KOH (pH 6-8.5). In addition to the above, 0.5 mM-5 mM of calcium chloride and 10 (v/v)%-50 (v/v)% of glycerol are preferably contained.

A reaction solution of cell-free protein synthesis system is prepared using, for example, an extract solution derived from arthropod or mammalian prepared in the manner, for example, as described above. Preferably, the reaction solution is prepared to contain 10 (v/v)%-80 (v/v)%, in particular, 30 (v/v)%-60 (v/v)% of the aforementioned extract solution. More specifically, in the whole reaction solution, the content of the extract derived from arthropod or mammalian culture cell is preferably 0.1 mg/mL-160 mg/mL in terms of protein concentration, more preferably 3 mg/mL-60 mg/mL. If the content of the extract is less than 0.1 mg/mL or exceeds 160 mg/mL in terms of protein concentration, the protein synthesis speed tends to deteriorate.

As far as the content of the extract falls within the aforementioned range, the extract solution derived from arthropod or derived from mammalian cell may be used alone, or mixture of different extract solutions. When different extract solutions are mixed, they may be mixed in any ratio.

In a reaction solution for translation system and a reaction solution for transcription/translation system using an extract solution containing an extract derived from arthropod, conventionally known components may be appropriately included without any specific limitation. In particular, in a reaction solution for translation system, components described in JP-A-2003-235598 in the case that a reaction solution for translation system is derived from silk worm tissue, and components described in JP-A-2004-215651 in the case that an extract solution is derived from culture cell, are preferably contained from the viewpoint of ability to synthesize a large amount of proteins in a short time. In the case of the reaction solution for transcription/translation system, for example, components described in JP-A-2003-245094 are contained.

Preferably, the reaction solution of cell-free protein synthesis system using an extract solution containing an extract derived from mammalian culture cell contains, as components besides the extract solution of the aforementioned mammalian culture cell, at least foreign mRNA, potassium salt, magnesium salt, DTT, adenosine triphosphate, guanosine triphosphate, creatine phosphate, creatine kinase, amino acid components and a buffer. By conducting translation reaction using such a reaction solution, it is possible to synthesize a large amount of proteins in a short time.

The foreign mRNA used in the reaction solution represents mRNA transcribed from a template DNA (a structural gene encoding a target protein is inserted into the expression vector of the present invention), and there is no specific limitation for an encoded protein (including peptide). It may encode a protein having toxity or a glycoprotein, or may be a base sequence encoding a fusion protein. From the viewpoint of facilitating purification of the expressed protein, a base sequence that encodes conventionally known histidine tag or GST tag may be added. These tag sequences are usually added to an N terminal or C terminal of the target protein.

As to the foreign mRNA used in the reaction solution, there is no specific limitation for its number of bases, and every mRNA does not necessarily have the same number of bases insofar as the target protein can be synthesized. Each mRNA may have deletion, substitution, insertion and addition of a plurality of bases insofar as it has such a homogenous sequence that allows synthesis of the target protein.

From the viewpoint of protein synthesis speed, in the reaction solution, the foreign mRNA is contained preferably in a proportion of 1 μg/mL-1000 μg/mL, more preferably in a proportion of 10 μg/mL-500 μg/mL. If the foreign mRNA is less than 1 μg/mL or exceeds 1000 μg/mL, the speed of protein synthesis tends to deteriorate.

As the potassium salt in the reaction solution, various potassium salts described above as a component of solution for extraction, preferably potassium acetate, can be preferably used. The potassium salt is preferably contained in the reaction solution in a proportion of 10 mM-500 mM, more preferably 20 mM-300 mM, from the same aspect of the potassium salt in the aforementioned solution for extraction.

As the magnesium salt in the reaction solution, various magnesium salt described above as a component of solution for extraction, preferably magnesium acetate, can be preferably used. The magnesium salt is preferably contained in the reaction solution in a proportion of 0.1 mM-10 mM, more preferably 0.5 mM-5 mM, from the same aspect of the magnesium salt in the aforementioned extract solution.

DTT is preferably contained in the reaction solution in a proportion of 0.1 mM-10 mM, more preferably 0.5 mM-5 mM, from the same aspect of DTT in the aforementioned solution for extraction.

The adenosine triphosphate (hereinafter sometimes to be referred to as “ATP”) in the reaction solution is preferably contained in the reaction solution in a proportion of 0.01 mM-10 mM, more preferably 0.1 mM-5 mM, in view of the rate of protein synthesis. When ATP is contained in a proportion of less than 0.01 mM or above 10 mM, the synthesis rate of the protein tends to become lower.

The guanosine triphosphate (hereinafter sometimes to be referred to as “GTP”) in the reaction solution preferably contained in the reaction solution in a proportion of 0.01 mM-10 mM, more preferably 0.05 mM-5 mM, in view of the rate of protein synthesis. When GTP is contained in a proportion of less than 0.01 mM or above 10 mM, the synthesis rate of the protein tends to become lower.

The creatine phosphate in the reaction solution is a component for continuous synthesis of protein and added for regeneration of ATP and GTP. The creatine phosphate is preferably contained in the reaction solution in a proportion of 1 mM-200 mM, more preferably 10 mM-100 mM, in view of the rate of protein synthesis. When creatine phosphate is contained in a proportion of less than 1 mM, sufficient amounts of ATP and GTP may not be regenerated easily. As a result, the rate of protein synthesis tends to become lower. When the creatine phosphate content exceeds 200 mM, it acts as an inhibitory substance and the rate of protein synthesis tends to become lower.

The creatine kinase in the reaction solution is a component for continuous synthesis of protein and added along with creatine phosphate for regeneration of ATP and GTP. The creatine kinase is preferably contained in the reaction solution in a proportion of 1 μg/mL-1000 μg/mL, more preferably 10 μg/mL-500 μg/mL, in view of the rate of protein synthesis. When the creatine kinase content is less than 1 μg/mL, sufficient amount of ATP and GTP may not be regenerated easily. As a result, the rate of protein synthesis tends to become lower. When the creatine kinase content exceeds 1000 μg/mL, it acts as an inhibitory substance and the synthesis rate of the protein tends to become lower.

The amino acid component in the reaction solution contains at least 20 kinds of amino acids, i.e., valine, methionine, glutamic acid, alanine, leuicine, phenylalanine, glycine, proline, isoleucine, tryptophan, asparagine, serine, threonine, histidine, aspartic acid, tyrosine, lysine, glutamine, cystine and arginine. This amino acid includes radioisotope-labeled amino acid. Where necessary, modified amino acid may be contained. The amino acid component generally contains almost the same amount of various kinds of amino acids.

In the present invention, the above-mentioned amino acid component is preferably contained in the reaction solution in a proportion of 1 μM-1000 μM, more preferably 10 μM-200 μM, in view of the rate of protein synthesis. When the amount of the amino acid component is less than 1 μM or above 1000 μM, the synthesis rate of the protein tends to become lower.

The buffer to be contained in the reaction solution is preferably similar to those used for the aforementioned extract solution of the present invention, and the use of HEPES-KOH (pH 6-8.5) is preferable for the same reasons. The buffer is preferably contained in the amount of 5 mM-200 mM, more preferably 10 mM-100 mM, from the same aspect as in the aforementioned buffer contained in the extract solution.

In the reaction solution, preferably, RNase inhibitor is further added. The RNase inhibitor is added to prevent RNase, which is derived from mammalian culture cells contaminating the extract solution, from undesirably digesting exogenous mRNA and tRNA, thereby preventing synthesis of protein, during cell-free protein synthesis system of the present invention. It is preferably contained in the reaction solution in a proportion of 0.1 U/μL-100 U/μL, more preferably 0.5 U/μL-10 U/μL.

In the reaction solution, preferably, tRNA is further added. The tRNA in the reaction solution contains almost the same amount of each of the tRNAs corresponding to the above-mentioned 20 kinds of amino acids. The tRNA is preferably contained in the reaction solution in a proportion of 1 μg/mL-1000 μg/mL, more preferably 10 μg/mL-500 μg/mL, in view of the rate of protein synthesis.

In the reaction solution, preferably, calcium salt is further added. As the calcium salt, various kinds of calcium salts as described for components of the solution for extraction, preferably, calcium chloride is used. From a similar viewpoint as is the case of the calcium salt in the above-described solution for extraction, the calcium salt is contained preferably in a proportion of 0.05 mM-10 mM, more preferably in a proportion of 0.1 mM-5 mM in the reaction solution.

Preferably, the reaction solution using an extract solution derived from mammalian culture cell is realized to contain 30 (v/v)%-60 (v/v)% of the extract solution, as well as 20 mM-300 mM of potassium acetate, 0.5 mM-5 mM of magnesium acetate, 0.5 mM-5 mM of DTT, 0.1 mM-5 mM of ATP, 0.05 mM-5 mM of GTP, 10 mM-100 mM of creatine phosphate, 10 μg/mL-500 μg/mL of creatine kinase, 10 μM-200 μM of amino acid components, 10 μg/mL-500 μg/mL of foreign mRNA, and 10 mM-100 mM of HEPES-KOH (pH 6-8.5). More preferably, the reaction solution is realized to further contain 0.5 U/μL-10 U/μL of RNase inhibitor, 10 μg/mL-500 μg/mL of tRNA, and 0.1 mM-5 mM of calcium chloride in addition to the above.

The cell-free protein synthesis system reaction using the reaction solution containing an extract solution derived from arthropod or extract solution derived from mammalian culture cell is carried out, in a conventionally known, for example, low-temperature incubator. The reaction temperature is usually within a range of 10° C.-40° C., preferably in a range of 20° C.-30° C. If the reaction temperature is less than 10° C., the protein synthesis speed tends to deteriorate, whereas if the reaction temperature exceeds 40° C., necessary components tend to denature. The reaction time is usually 1 hour-72 hours, preferably 3 hours-24 hours.

In the case of cell-free protein synthesis system using a reaction solution containing an extract solution derived from mammalian culture cell, it is preferable to conduct a certain period of incubation in the condition that other components in the composition of the reaction solution than mRNA are added to the extract solution, prior to conducting the synthesis reaction. The incubation is conducted in a conventionally known, for example, low-temperature incubator. The incubation time is usually 0° C.-50° C., preferably 15° C.-37° C. If the incubation temperature is less than 0° C., the effect of incubation is difficult to be obtained, whereas if the incubation temperature exceeds 50° C., necessary components tend to denature. The period of incubation is usually 1 minute-120 minutes, preferably 10 minutes-60 minutes.

As to the cell-free protein synthesis reaction (transcription/translation system synthesis reaction) using the reaction solution for transcription/translation system, it may be conducted in a conventionally known, for example, low-temperature incubator as is the case of the aforementioned translation system synthesis reaction. The reaction temperature in the transcription step is usually in a range of 10° C.-60° C., preferably in a range of 20° C.-50° C. If the reaction temperature in the transcription step is less than 10° C., the transcription speed tends to deteriorate, whereas if the reaction temperature in the transcription step exceeds 60° C., components essential for the reaction tend to denature. The temperature in the translation step is usually in a range of 10° C.-40° C., preferably in a range of 20° C.-30° C. If the reaction temperature in the translation step is less than 10° C., the protein synthesis speed tends to deteriorate, whereas if the reaction temperature in the translation step exceeds 40° C., components essential for the reaction tend to denature.

In synthesis reaction based on the transcription/translation system, it is particularly preferable to conduct the reaction at a temperature in a range of 20-30° C. which is preferable for both steps from the viewpoint of possibility of continuous executions of the transcription step and the translation step. The reaction time for the entire process is usually 1 hour-72 hours, preferably 3 hours-24 hours.

There is no specific limitation for the proteins that can be synthesized using the aforementioned reaction solution for translation system and reaction solution for transcription/translation system. The amount of synthesized protein may be determined by measurement of enzymatic activity, SDS-PAGE, immunoassay and the like.

<Kit for Cell-Free Protein Synthesis System>

The present invention also provides a cell-free synthesis system kit including the expression vector of the present invention. The cell-free protein synthesis system kit includes a cell-free protein synthesis system reaction solution. Preferred expression vector, composition, concentration and other preferred composition of the cell-free protein synthesis system reaction solution in the cell-free protein synthesis kit are as described above. The cell-free protein synthesis system kit is not particularly limited insofar as it comprises an appropriate container accommodating an expression vector and a cell-free protein synthesis system reaction solution and other appropriate elements. The extraction used for the cell-free protein synthesis system may be accommodated separately from the reaction solution for cell-free protein synthesis system from the viewpoint of storage.

### EXAMPLES

In the following, the present invention will be explained in more detail by way of examples, however, the present invention is not limited to the examples provided below.

### Reference Example 1

### Construction of vector pTNT-Luc

Using 5 ng of pGEM-Luc Vector (manufactured by Promega Corporation) having a structural gene encoding luciferase as a template, and a primer having a base sequence represented by SEQ ID No. 12 of the sequence listing (LucT7-F3-Kpn) and a primer having a base sequence represented by SEQ ID No. 13 of the sequence listing (Luc T7-R4-Kpn) and KOD plus (manufactured by TOYOBO Co., Ltd.), denaturing the template at 96° C. for 2 minutes and then 30 cycles (each cycle includes 96° C. 15 seconds, 50° C. 30 seconds and 68° C. 120 seconds) of polymerase chain reaction (PCR) was conducted to amplify the open reading frame (ORF) of the structural gene. The PCR product was purified by ethanol precipitation and then digested with KpnI. Separately from this, pTNT Vector (manufactured by Promega Corporation) was digested with KpnI. These reaction solutions were separated by agarose gel electrophoresis and then purified by using Gen Elute Gel Purification Kit (manufactured by SIGMA Corporation). The resultant reaction solutions were ligated using Ligation High (manufactured by TOYOBO Co., Ltd.), and E. coli DH5α (manufactured by TOYOBO Co., Ltd.) was transformed. A plasmid prepared from the transformed E. coli by Alkaline-SDS method was subjected to a sequencing reaction (96° C. 10 seconds, 50° C. 5 seconds and 60° C. 4 minutes, 25 cycles) using a primer having a base sequence represented by SEQ ID No. 14 of the sequence listing (T7 promoter) and Big Dye Terminator Cycle Sequencing FS (manufactured by Applied Biosystems). The resultant reaction mixture was applied to an ABI PRISM 310 Genetic Analyzer (manufactured by Applied Biosystems) for analysis of base sequence. The plasmid in which an initiation codon of luciferase gene is incorporated downstream side of the 5′-β globin leader sequence derived from pTNT Vector was named “pTNT-Luc”.

### Reference Example 2

### Production of Template DNA (Vector pFib-Luc)

Using the plasmid vector pTNT-Luc produced in Reference Example 1 as a template, and a primer having a base sequence represented by SEQ ID No. 15 of the sequence listing (T7p Rv) and a primer having a base sequence represented by SEQ ID No. 16 of the sequence listing (Luc-ATG), 30 cycles (each cycle including 96° C. 15 seconds, 50° C. 30 seconds, 68° C. 5 minutes) of PCR was conducted. After completion of the reaction, the PCR product was separated by electrophoresis, and purified using Gen Elute Gel Purification Kit (manufactured by SIGMA Corporation), and the resultant product was used for ligation reaction. In this manner, a plasmid vector in which SP6 promoter sequence, 5′-β globin leader sequence and multi-cloning site on the upstream side of 5′ of structural gene encoding luciferase are deleted from the plasmid vector pTNT-Luc was obtained for examining the effect of insertion of 5′UTR.

A sense strand and an anti-sense strand of 5′UTR of fibroin L-chain gene of silk worm having a base sequence represented by SEQ ID No. 1 of the sequence listing were synthesized by a DNA synthesizer, and 5′ terminals thereof were phosphorylated using T4 Polynucleotide Kinase (manufactured by TOYOBO Co., Ltd.). After completion of reaction, the sense strand and the anti-sense strand were mixed and heated at 95° C. for 5 minutes. The mixture was allowed to reach room temperature to make the sense strand and the anti-sense strand anneal to each other. After purification by ethanol precipitation, the products were dissolved in water. After removing excess ATP by using Sigma Spin Post Reaction purification Columns (manufactured by SIGMA Corporation), purification by ethanol precipitation was conducted again. Using the resultant double-stranded DNA fragment as an insert, the insert was ligated into the vector derived from pTNT-Luc and lacking SP6 promoter sequence, β globin leader sequence and multi-cloning site on the upstream side of 5′ of structural gene, and E. coli DH5α was transformed. After preparing a plasmid from the obtained E. coli, a sequence analysis was conducted. In this manner, a vector in which one 5′UTR of fibroin L chain gene of silk worm was incorporated in forward direction (5′→3′) was selected. In this way, a vector (template DNA) in which one 5′UTR of fibroin L chain gene of silk worm was incorporated in forward direction between the T7 promoter sequence and the structural gene was produced. The obtained template DNA was named “pFib-Luc”.

### Reference Example 3

### Production of Template DNA (Vector pSer-Luc)

In the same manner as Reference Example 2 except that 5′UTR of sericin gene of silk worm having a base sequence represented by SEQ ID No. 2 of the sequence listing was used, a vector (template DNA) in which one 5′UTR of sericin gene of silk worm having a base sequence represented by SEQ ID No. 2 of the sequence listing was incorporated in forward direction (5′→3′) between the T7 promoter sequence and the structural gene was produced. The obtained template DNA was named “pSer-Luc”.

### Reference Example 4

### Production of Template DNA (Vector pAphd-Luc)

In the same manner as Reference Example 2 except that 5′UTR of polyhedrin gene of AcNPV (Autographa californica nuclear polyhedrosis virus) having a base sequence represented by SEQ ID No. 3 of the sequence listing was used, a vector (template DNA) in which one 5′UTR of polyhedrin gene of AcNPV having a base sequence represented by SEQ ID No. 3 of the sequence listing was incorporated in forward direction (5′→3′) between the T7 promoter sequence and the structural gene was produced. The obtained template DNA was named “pAphd-Luc”.

### Reference Example 5

### Production of Template DNA (Vector pFAphd-Luc)

Both of the double-stranded DNA fragment prepared from 5′UTR of fibroin L-chain gene of silk worm in Reference Example 2 and the double-stranded DNA fragment prepared from 5′UTR of polyhedrin gene of ACNPV (Autographa californica nuclear polyhedrosis virus) in Reference Example 4 were used as inserts and ligation with the aforementioned pTNT-Luc-derived vector lacking SP6 promoter sequence, β globin leader sequence and multi-cloning site on the upstream side of 5′ of structural gene was conducted and E. coli DH5α was transformed. After preparing a plasmid from the obtained E. coli, base sequence analysis was conducted. Reference Example 2 was followed except that a vector (template DNA) in which each one of 5′UTR of fibroin L-chain gene of silk worm and 5′UTR of polyhedrin gene of AcNPV were sequentially incorporated from 5′ side in forward direction (5′→3′) was selected in the manner as described above. The obtained template DNA was named “pFAphd-Luc”.

### Reference Example 6

### Production of Template DNA (Vector pBphd-Luc)

In the same manner as Reference Example 2 except that 5′UTR of polyhedrin gene of BmCPV (Bombyx mori cytoplasmic polyhedrosis virus) having a base sequence represented by SEQ ID No. 4 of the sequence listing was used, a vector (template DNA) in which one 5′UTR of polyhedrin gene of BmCPV having a base sequence represented by SEQ ID No. 4 of the sequence listing was incorporated in forward direction (5′→3′) between the T7 promoter sequence and the luciferase gene was produced. The obtained template DNA was named “pBphd-Luc”.

### Reference Example 7

### Production of Template DNA (Vector pBphd-R-Luc)

Reference Example 6 was followed except that a vector (template DNA) in which one 5′UTR of polyhedrin gene of BmCPV was incorporated in reverse direction (3′->5′) was selected. The obtained template DNA was named “pBphd-R-Luc”.

### Reference Example 8

### Production of Template DNA (Vector pEphd-FF-Luc)

Using as an insert a double-stranded DNA fragment prepared in the same manner as Reference Example 2 except that 5′UTR of polyhedrin gene of EsCPV (Euxoa scandes cytoplasmic polyhedrosis virus) having a base sequence represented by SEQ ID No. 5 of the sequence listing was used, the ligation with the aforementioned pTNT-Luc-derived vector lacking SP6 promoter sequence, β globin leader sequence and multi-cloning site on the upstream side of 5′ of structural gene and conducted and E. coli DH5a was transformed. After preparing a plasmid from the obtained E. coli, base sequence analysis was conducted. Reference Example 2 was followed except that a vector (template DNA) in which two 5′UTRs of polyhedrin gene of EsCPV were sequentially incorporated from 5′ side in forward direction (5′→3′) was selected in the manner as described above. The obtained template DNA was named “pEphd-FF-Luc”.

### Reference Example 9

### Production of Template DNA (Vector pEphd-RR-Luc)

Reference Example 8 was followed except that a vector (template DNA) in which two 5′UTRs of polyhedrin gene of EsCPV were sequentially incorporated from 5′ side in reverse direction (3′→5′) was selected. The obtained template DNA was named “pEphd-RR-Luc”.

### Reference Example 10

### Production of Template DNA (Vector pHphd-Luc)

Using as an insert a double-stranded DNA fragment prepared in the same manner as Reference Example 2 except that 5′UTR of polyhedrin gene of HcNPV (Hyphantria cunea nuclear polyhedrosis virus) having a base sequence represented by SEQ ID No. 6 of the sequence listing was used, the ligation into the aforementioned pTNT-Luc-derived vector lacking SP6 promoter sequence, β globin leader sequence and multi-cloning site on the upstream side of 5′ of structural gene was conducted and E. coli DH5α was transformed. After preparing a plasmid from the obtained E. coli, base sequence analysis was conducted. Reference Example 2 was followed except that a vector (template DNA) in which one 5′UTR of polyhedrin gene of HcNPV was incorporated in forward direction (5′→3′) was selected in the manner as described above. The obtained template DNA was named “pHphd-Luc”.

### Reference Example 11

### Production of Template DNA (Vector pHphd-R-Luc)

Reference Example 10 was followed except that a vector (template DNA) in which one 5′UTR of polyhedrin gene of HcNPV was incorporated in reverse direction (3′→5′) was selected. The obtained template DNA was named “pHphd-R-Luc”.

### Reference Example 12

### Production of Template DNA (Vector pHphd-RR-Luc)

Reference Example 10 was followed except that a vector (template DNA) in which two 5′UTRs of polyhedrin gene of HcNPV were sequentially incorporated from 5′ side in reverse direction (3′→5′) was selected. The obtained template DNA was named “pHphd-RR-Luc”.

### Reference Example 13

### Production of Template DNA (Vector pCphd-Luc)

Using as an insert a double-stranded DNA fragment prepared in the same manner as Reference Example 2 except that 5′UTR of polyhedrin gene of CrNPV (Choristoneura rosaceana nucleopolyhedrovirus) having a base sequence represented by SEQ ID No. 7 of the sequence listing was used, the ligation into the aforementioned pTNT-Luc-derived vector lacking SP6 promoter sequence, β globin leader sequence and multi-cloning site on the upstream side of 5′ of structural gene was conducted and E. coli DH5α was transformed. After preparing a plasmid from the obtained E. coli, base sequence analysis was conducted. Reference Example 2 was followed except that a vector (template DNA) in which one 5′UTR of polyhedrin gene of CrNPV was incorporated in forward direction (5′→3′) was selected in the manner as described above. The obtained template DNA was named “pCphd-Luc”.

### Reference Example 14

### Production of Template DNA (Vector pCphd-R-Luc)

Reference Example 13 was followed except that a vector (template DNA) in which one 5′UTR of polyhedrin gene of CrNPV was incorporated in reverse direction (3′->5′) was selected. The obtained template DNA was named “pCphd-R-Luc”.

### Reference Example 15

### Production of Template DNA (Vector pEophd-R-Luc)

Using as an insert a double-stranded DNA fragment prepared in the same manner as Reference Example 2 except that 5′UTR of polyhedrin gene of EoNPV (Ecotropis oblique nuclear polyhedrosis virus) having a base sequence represented by SEQ ID No. 8 of the sequence listing was used, the ligation into the aforementioned pTNT-Luc-derived vector lacking SP6 promoter sequence, β globin leader sequence and multi-cloning site on the upstream side of 5′ of structural gene was conducted and E. coli DH5α was transformed. After preparing a plasmid from the obtained E. coli, base sequence analysis was conducted. Reference Example 2 was followed except that a vector (template DNA) in which one 5′UTR of polyhedrin gene of EoNPV was incorporated in reverse direction (3′→5′) was selected in the manner as described above. The obtained template DNA was named “pEophd-R-Luc”.

### Reference Example 16

### Production of Template DNA (Vector pMphd-FF-Luc)

Using as an insert a double-stranded DNA fragment prepared in the same manner as Reference Example 2 except that 5′UTR of polyhedrin gene of MnNPV (Malacosma neustria nucleopolyhedrovirus) having a base sequence represented by SEQ ID No. 9 of the sequence listing was used, the ligation into the aforementioned pTNT-Luc-derived vector lacking SP6 promoter sequence, β globin leader sequence and multi-cloning site on the upstream side of 5′ of structural gene was conducted and E. coli DH5α was transformed. After preparing a plasmid from the obtained E. coli, base sequence analysis was conducted. Reference Example 2 was followed except that a vector (template DNA) in which two 5′UTRs of polyhedrin gene of MnNPV were incorporated in forward direction (5′→3′) was selected in the manner as described above. The obtained template DNA was named “pMphd-FF-Luc”.

### Reference Example 17

### Production of Template DNA (Vector pMphd-R-Luc)

Reference Example 16 was followed except that a vector (template DNA) in which one 5′UTR of polyhedrin gene of MnNPV was incorporated in reverse direction (3′→5′) was selected. The obtained template DNA was named “pMphd-R-Luc”.

### Reference Example 18

### Production of Template DNA (Vector pSphd-Luc)

Using as an insert a double-stranded DNA fragment prepared in the same manner as Reference Example 2 except that 5′UTR of polyhedrin gene of SfNPV (Spodoptera frugiperda nucleopolyhedrovirus) having a base sequence represented by SEQ ID No. 10 of the sequence listing was used, the ligation into the aforementioned pTNT-Luc-derived vector lacking SP6 promoter sequence, β globin leader sequence and multi-cloning site on the upstream side of 5′ of structural gene was conducted and E. coli DH5α was transformed. After preparing a plasmid from the obtained E. coli, base sequence analysis was conducted. Reference Example 2 was followed except that a vector (template DNA) in which one 5′UTR of polyhedrin gene of SfNPV was incorporated in forward direction (5′→3′) was selected in the manner as described above. The obtained template DNA was named “pSphd-Luc”.

### Reference Example 19

### Production of Template DNA (Vector pWphd-Luc)

Using as an insert a double-stranded DNA fragment prepared in the same manner as Reference Example 2 except that 5′UTR of polyhedrin gene of WsNPV (Wiseana signata nucleopolyhedrovirus) having a base sequence represented by SEQ ID No. 11 of the sequence listing was used, the ligation into the aforementioned pTNT-Luc-derived vector lacking SP6 promoter sequence, β globin leader sequence and multi-cloning site on the upstream side of 5′ of structural gene was conducted and E. coli DH5α was transformed. After preparing a plasmid from the obtained E. coli, base sequence analysis was conducted. Reference Example 2 was followed except that a vector (template DNA) in which one 5′UTR of polyhedrin gene of WsNPV was incorporated in forward direction (5′→3′) was selected in the manner as described above. The obtained template DNA was named “pWphd-Luc”.

### Reference Example 20

### Preparation of Extract Solution of Insect Culture Cell (High Five)

(1) Cultivation of Insect Culture Cell

2.1×10⁷ of insect culture cell High Five (manufactured by Invitrogen Corporation) were cultured in a cultivation flask (600 cm²) containing Express Five serum-free medium (manufactured by Invitrogen Corporation) supplemented with L-glutamine at 27° C. for 6 days. After cultivation, the number of cells were 1.0×10⁸, and wet weight was 1.21 g.

(2) Preparation of Extract Solution of Insect Culture Cell

First, the insect culture cells cultured in the above (1) were collected and washed (centrifugation at 700×g, 4° C., 10 minutes) three times with a washing solution having the following composition.

[Composition of Washing Solution]

60 mM HEPES-KOH (pH 7.9)

200 mM potassium acetate

4 mM magnesium acetate

4 mM DTT

To the insect culture cell after washing, 1 mL of a solution for extraction having the following composition was added and suspended.

[Composition of Solution for Extraction]

40 mM HEPES-KOH (pH 7.9)

100 mM potassium acetate

2 mM magnesium acetate

2 mM calcium chloride

20 (v/v)% glycerol

1 mM DTT

1 mM PMSF

The resultant suspension was rapidly frozen in liquid nitrogen. After having sufficiently frozen, the suspension was thawed in an ice water bath at about 4° C. After having completely thawed, centrifugation (himacCR20B3, manufactured by Hitachi Koki Co., Ltd.) at 30,000×g, 4° C. for 10 minutes was followed, and the supernatant was collected. 1.5 mL of the collected supernatant was applied to a PD-10 desalted column (manufactured by Amersham Biosciences) equilibrated with a buffer for gel filtration having the following composition.

[Composition of Buffer for Gel Filtration]

40 mM HEPES-KOH (pH 7.9)

100 mM potassium acetate

2 mM magnesium acetate

1 mM DTT.

1 mM PMSF

After application, elution with 4 mL of buffer for gel filtration was followed, and fractions having an absorbance of 30 or more at 280 nm measured by a spectrometer (Ultrospec3300pro, manufactured by Amersham Biosciences) were collected, to give an extract solution of insect culture cell.

### Reference Example 21

### Preparation of Extract Solution of Insect Culture Cell (Sf21)

(1) Cultivation of Insect Culture Cell

Insect cells Sf21 (manufactured by Invitrogen Corporation) were cultured in Sf900-II serum-free medium (manufactured by Invitrogen Corporation) at 27° C. 6.0×10⁵ Sf21 cells per 1 mL of medium was subjected to suspension culture in 50 mL of medium in a 125-mL Erlenmeyer flask at 27° C., 130 rpm for 5 days. As a result, the number of cells per 1 mL of medium was 1.0×10⁸ and wet weight was 3 g. Using these cells, an extract solution of cell was prepared.

(2) Preparation of Extract Solution of Insect Culture Cell

First, the insect culture cells cultured in the above (1) were collected and washed (centrifugation at 700×g, 4° C., 10 minutes) three times with a washing solution having the following composition.

[Composition of Washing Solution]

40 mM HEPES-KOH (pH 7.9)

100 mM potassium acetate

2 mM magnesium acetate

2 mM calcium chloride

1 mM DTT

To the insect culture cell after washing, 3 mL of a solution for extraction having the following composition was added and suspended.

[Composition of Solution for Extraction]

40 mM HEPES-KOH (pH 7.9)

100 mM potassium acetate

2 mM magnesium acetate

2 mM calcium chloride

20 (v/v)% glycerol

1 mM DTT

0.5 mM PMSF

The resultant suspension was rapidly frozen in liquid nitrogen. After having sufficiently frozen, the suspension was thawed in an ice water bath at about 4° C. After having completely thawed, centrifugation (himacCR20B3, manufactured by Hitachi Koki Co., Ltd.) at 30,000×g, 4° C. for 10 minutes was followed, and the supernatant was collected. The collected supernatant was further centrifuged at 45,000×g, 4° C. for 30 minutes, and the supernatant was collected. 2.5 mL of the collected supernatant was applied to a PD-10 desalted column (manufactured by Amersham Biosciences) equilibrated with a buffer for gel filtration having the following composition.

[Composition of Buffer for Gel Filtration]

40 mM HEPES-KOH (pH 7.9)

100 mM potassium acetate

2 mM magnesium acetate

1 mM DTT

0.5 mM PMSF

After application, elution with 3 mL of buffer for gel filtration was followed, and fractions having an absorbance of 30 or more at 280 nm measured by a spectrometer (Ultrospec3300pro, manufactured by Amersham Biosciences) were collected, to give an extract solution of insect culture cell.

### Reference Example 22

### Preparation of Silk Worm Extract Solution

From 15 young silkworms at fourth day in the fifth period, 3.07 g of posterior silk gland was removed by means of scissors, tweezers, surgical knife and spatula, grinded in a frozen mortar at −80° C., and then extracted using a solution for extraction having the following composition.

[Composition of Solution for Extraction]

20 mM HEPES-KOH (pH 7.4)

100 mM potassium acetate

2 mM magnesium acetate

2 mM DTT

0.5 mM PMSF

After extraction, the obtained liquid-like product was centrifuged by a centrifugal separator (himac CR20B3 (manufactured by Hitachi Koki CO., Ltd.)) in the condition of 30,000×g, 30 minutes and 4° C.

After centrifugation, the supernatant was solely isolated, and centrifuged again in the condition of 30,000×g, 10 minutes and 4° C. After centrifugation, the supernatant was solely isolated. After equilibrating a desalted column PD-10 (manufactured by Amersham Biosciences) by adding a solution for extraction containing 20% glycerol, the supernatant was applied on the column and subjected to gel filtration through elution with the above solution for extraction.

From filtrate fractions after gel filtration, a fraction that had absorbance of 10 or more at 280 nm was collected using a spectrometer (Ultrospec3300pro, manufactured by Amersham Biosciences), to give an extract solution for cell-free protein synthesis system derived from posterior silk gland of young silk worm in the fifth period.

### Reference Example 23

### Preparation of Mammalian Culture Cell (CHO) Extract Solution

(1) Cultivation of Mammalian Culture Cell

Chinese hamster ovary cells CHO K1-SFM (obtained from the Cancer Cell Repository, Institute of Development, Aging and Cancer, Tohoku University) at a cell concentration of 4.9×10⁵ cells/mL were cultured in 200 mL of CHO SERUM-FREE MEDIUM (manufactured by SIGMA Corporation) contained in an Erlenmeyer flask (500 mL) for 120 hours at 130 rpm, 37° C., under 5% CO₂ atmosphere. As a result, the cell concentration was 8.8×10⁶ cells/mL, and the wet weight was 3.2 g.

(2) Preparation of Extract Solution of Mammalian Culture Cell (CHO)

First, the animal culture cells cultured in the above (1) were collected by centrifugal separation (700×g, 10 minutes) and washed three times (centrifuged in the condition of 700×g, 10 minutes) with a buffer for washing having the following composition.

[Composition of Buffer for Washing]

40 mM HEPES-KOH (pH 7.9)

100 mM potassium acetate

2 mM magnesium acetate

2 mM calcium chloride

1 mM DTT

To the mammalian culture cells after washing, 0.8 mL per 1 g of wet cell weight of a solution for extraction having the following composition was added and cells were suspended.

[Composition of Solution for Extraction]

40 mM HEPES-KOH (pH 7.9)

100 mM potassium acetate

2 mM magnesium acetate

2 mM calcium chloride

20 (v/v)% glycerol

1 mM DTT

This suspension was rapidly frozen in liquid nitrogen. After having sufficient frozen, the suspension was thawed in ice water bath at about 4° C. After having completely thawed, centrifugal separation at 30,000×g, 4° C. was conducted for 10 minutes (by himacCR20B3, manufactured by Hitachi Koki Co., Ltd.) and the supernatant was collected. The collected supernatant was further centrifuged at 30,000×g, 4° C. for 30 minutes and the supernatant was collected. 2.0 mL of the collected supernatant was applied to a desalted column PD-10 (manufactured by Amersham Biosciences) having equilibrated with a buffer for gel filtration having the following composition.

[Composition of Buffer for Gel Filtration]

40 mM HEPES-KOH (pH 7.9)

100 mM potassium acetate

2 mM magnesium acetate

1 mM DTT

Following the application, elution with 3 mL of buffer for gel filtration was followed, and fractions having an absorbance of 30 or more at 280 nm measured by a spectrometer (Ultrospec3300pro, manufactured by Amersham Biosciences) were collected, to give an extract solution of mammalian culture cell.

### Experiment Example 1

### In Vitro Transcription Reaction and Purification of mRNA

Each of the vectors (template DNAs) produced in Reference Examples 1-19 was digested with BamHI or BglII, and extracted with phenol/chloroform and the purified by ethanol precipitation. 1 μg of the obtained vector was used as a template, and mRNA was synthesized by conducting in vitro transcription reaction at 37° C. for 4 hours in 20 μL scale using RiboMax Large Scale RNA production System-T7 (manufactured by Promega Corporation).

After completion of the transcription reaction, 1 U of RQ1 RNase Free DNase (manufactured by Promega Corporation) was added, and incubated at 37° C. for 15 minutes to digest the template. After removing proteins by phenol/chloroform extraction, ethanol precipitation was conducted. The obtained precipitation was dissolved in 100 μL of sterilized water, applied on a Nick column (manufactured by Amersham Biosciences), and the eluted with sterilized water. To the eluted fraction, potassium acetate was added in a final concentration of 0.3 M and ethanol precipitation was conducted. Quantification of the synthesized mRNA was conducted by measuring absorbances at 260 nm and 280 nm.

### Experiment Example 2

### Cell-Free Protein Synthesis System Using Reaction Solution for Translation System Containing Extract Derived from Insect Culture Cell (High Five)

For each of the mRNAs prepared in the manner as described in Experiment Example 1 from the template DNAs produced in Reference Examples 2-7, 9 and 11-19, using the extract solution of insect cell prepared in Reference Example 20, translation reaction was conducted by preparing a reaction solution for translation system having the following composition.

[Composition of Reaction Solution for Translation System]

50 (v/v)% insect culture cell extract solution

320 μg/mL mRNA

30 mM HEPES-KOH (pH 7.9)

100 mM potassium acetate

2 mM magnesium acetate

2 mM DTT

0.5 mM ATP

0.25 mM GTP

20 mM creatine phosphate

200 μg/mL creatine kinase

40 μM amino acids (20 kinds)

0.25 mM EGTA

1 U/μL RNase inhibitor (from human placenta)

200 μg/mL tRNA

ATP (manufactured by SIGMA Corporation), GTP (manufactured by SIGMA Corporation), 20 kinds of amino acids (manufactured by SIGMA Corporation), RNase inhibitor (manufactured by Takara Shuzo Co., Ltd.) and tRNA (manufactured by Roche Diagnostics Co., Ltd.) were respectively used.

As a reaction device, a low-temperature aluminum block incubator MG-1000 (manufactured by Tokyo Rikakikai Co., Ltd.) was used. Translation reaction was conducted at a reaction temperature of 25° C. for 5 hours, and the amount of reaction solution was 25 μL.

Synthesized luciferase was quantified by using luciferase assay kit (E-1500, manufactured by Promega Corporation). To 50 μL of luciferase assay reagent, 2.5 μL of reaction solution was added, and light emission by luciferase was measured by using a luminometer (Tuner Designs TD-20/20, manufactured by Promega Corporation).

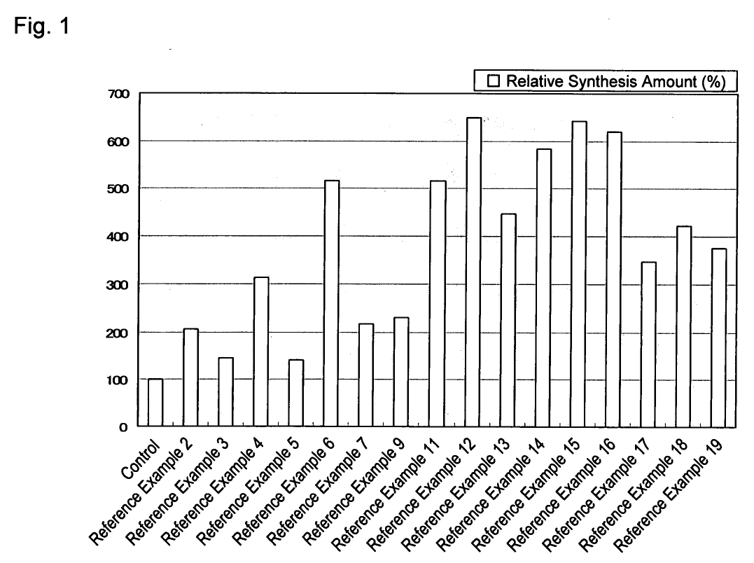

FIG. 1 is a graph showing a result of Experiment Example 2, in which a relative synthesis amount to luciferase control RNA (manufactured by Promega Corporation) (100%) is represented on the vertical axis.

### Experiment Example 3

### Cell-Free Protein Synthesis System using Reaction Solution for Translation System Containing Extract Derived from Insect Culture Cell (Sf21)

For each of the mRNAs prepared in the manner as described in Experiment Example 1 from the template DNAs produced in Reference Examples 2-4, 6-16, 18 and 19, using the extract solution of insect cell prepared in Reference Example 21, translation reaction was conducted by preparing a reaction solution for translation system having the following composition.

[Composition of Reaction Solution for Translation System]

50 (v/v)% insect culture cell extract solution

320 μg/mL mRNA

40 mM HEPES-KOH (pH 7.9)

100 mM potassium acetate

1.5 mM magnesium acetate

2 mM DTT

0.25 mM ATP

0.1 mM GTP

20 mM creatine phosphate

200 μg/mL creatine kinase

80 μM amino acids (20 kinds)

0.1 mM EGTA

1 U/μL RNase inhibitor (derived from human placenta)

200 μg/mL tRNA

ATP (manufactured by SIGMA Corporation), GTP (manufactured by SIGMA Corporation), 20 kinds of amino acids (manufactured by SIGMA Corporation), RNase inhibitor (manufactured by Takara Shuzo Co., Ltd.) and tRNA (manufactured by Roche Diagnostics Co., Ltd.) were respectively used.

As a reaction device, a low-temperature aluminum block incubator MG-1000 (manufactured by Tokyo Rikakikai Co., Ltd.) was used. Translation reaction was conducted at a reaction temperature of 25° C. for 5 hours, and the amount of reaction solution was 25 μL.

Synthesized luciferase was quantified by using luciferase assay kit (E-1500, manufactured by Promega Corporation). To 50 μL of luciferase assay reagent, 2.5 μL of reaction solution was added, and light emission by luciferase was measured by using a luminometer (Tuner Designs TD-20/20, manufactured by Promega Corporation).

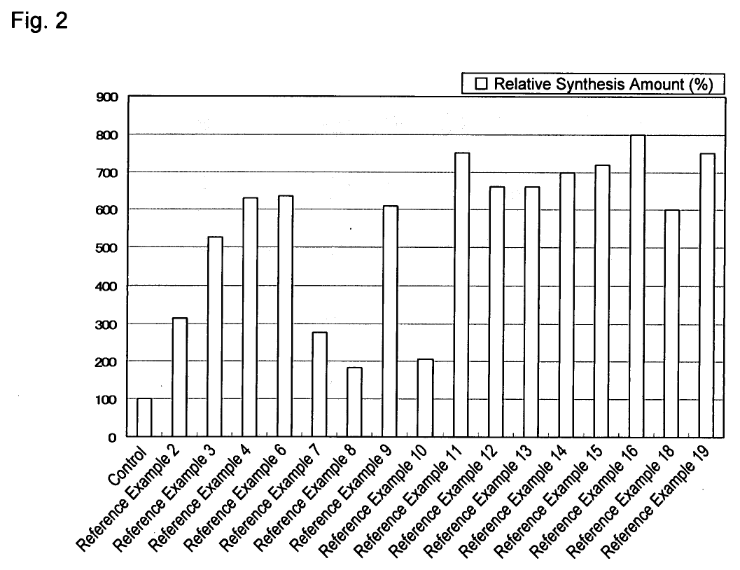

FIG. 2 is a graph showing a result of Experiment Example 3, in which a relative synthesis amount to luciferase control RNA (manufactured by Promega Corporation) (100%) is represented on the vertical axis.

### Experiment Example 4

### Cell-Free Protein Synthesis System using Reaction Solution for Translation System Containing Extract Derived from Silk Worm Tissue

For each of the mRNAs prepared in the manner as described in Experiment Example 1 from the template DNAs produced in Reference Examples 2-19, using the silk worm extract solution prepared in Reference Example 22, translation reaction was conducted by preparing a reaction solution for translation system having the following composition.

[Composition of Reaction Solution for Translation System]

50 (v/v)% silk worm extract solution

160 μg/mL mRNA

30 mM HEPES-KOH (pH 7.4)

100 mM potassium acetate

1.0 mM magnesium acetate

0.5 mM DTT

10 (v/v)% glycerol

0.5 mM ATP

0.5 mM GTP

0.25 mM EGTA

25 mM creatine phosphate

200 μg/mL creatine kinase

40 μM amino acids (20 kinds)

2 U/μL RNase inhibitor

200 μg/mL tRNA

ATP (manufactured by SIGMA Corporation), GTP (manufactured by SIGMA Corporation), 20 kinds of amino acids (manufactured by SIGMA Corporation), RNase inhibitor (manufactured by Takara Shuzo Co., Ltd.) and tRNA (manufactured by Roche Diagnostics Co., Ltd.) were respectively used. As a foreign mRNA, mRNA encoding luciferase (luciferase control RNA, manufactured by Promega Corporation) was used.

As a reaction device, a low-temperature aluminum block incubator MG-1000 (manufactured by Tokyo Rikakikai Co., Ltd.) was used. Translation reaction was conducted at a reaction temperature of 25° C. for 5 hours, and the amount of reaction solution was 25 μL. Synthesized luciferase was quantified by using luciferase assay kit (E-1500, manufactured by Promega Corporation). To 50 μL of luciferase assay reagent, 2.5 μL of reaction solution was added, and light emission by luciferase was measured by using a luminometer (Tuner Designs TD-20/20, manufactured by Promega Corporation).

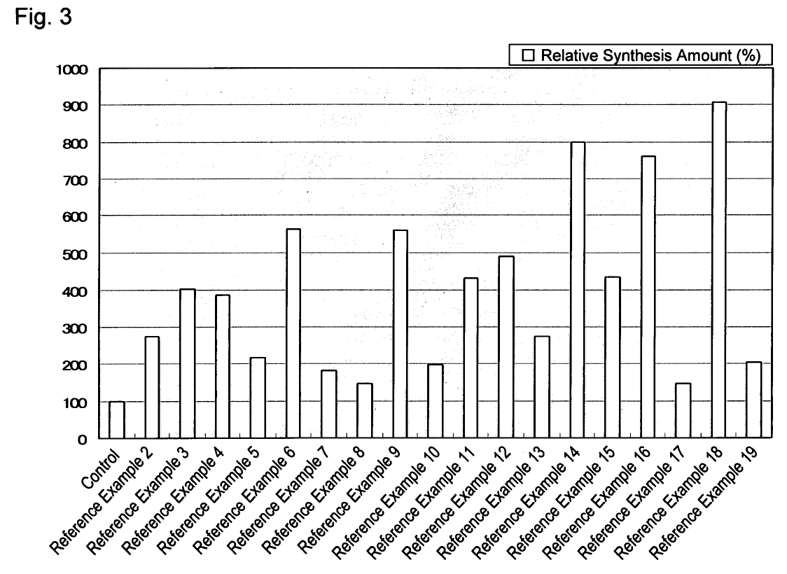

FIG. 3 is a graph showing a result of Experiment Example 4, in which a relative synthesis amount to luciferase control RNA (manufactured by Promega Corporation) (100%) is represented on the vertical axis.

### Experiment Example 5

### Cell-Free Protein Synthesis System Using Reaction Solution for Translation System Containing Extract Derived from Mammalian Culture Cell (CHO)

For each of the mRNAs prepared in the manner as described in Experiment Example 1 from the template DNAs produced in Reference Examples 1, 5, 6, 8, 11, 12, 14, 15, 16, 18 and 19, using the extract solution of mammalian culture cell prepared in Reference Example 23, translation reaction was conducted by preparing a reaction solution for translation system having the following composition.

[Composition of Reaction Solution for Translation System]

50 (v/v)% mammalian culture cell extract solution

160 μg/mL mRNA

50 mM HEPES-KOH (pH 7.9)

175 mM potassium acetate

1 mM magnesium acetate

0.5 mM calcium chloride

2 mM DTT

0.5 mM ATP

0.25 mM GTP

30 mM creatine phosphate

200 μg/mL creatine kinase

80 μM amino acid (20 kinds)

0.25 mM EGTA

ATP (manufactured by SIGMA Corporation), GTP (manufactured by SIGMA Corporation) and 20 kinds of amino acids (manufactured by SIGMA Corporation) were respectively used.

As a reaction device, a low-temperature aluminum block incubator MG-1000 (manufactured by Tokyo Rikakikai Co., Ltd.) was used. For starting translation reaction, first, a reaction solution for translation not containing mRNA in the aforementioned reaction solution for translation was prepared, and incubated at 25° C. for 30 minutes. Then mRNA was added and translation reaction was started (25° C. for 4 hours). The amount of reaction solution was 25 μL.

Synthesized luciferase was quantified by using luciferase assay kit (E-1500, manufactured by Promega Corporation). To 50 μL of luciferase assay reagent, 2.5 μL of reaction solution was added, and light emission by luciferase was measured by using a luminometer (Tuner Designs TD-20/20, manufactured by Promega Corporation).

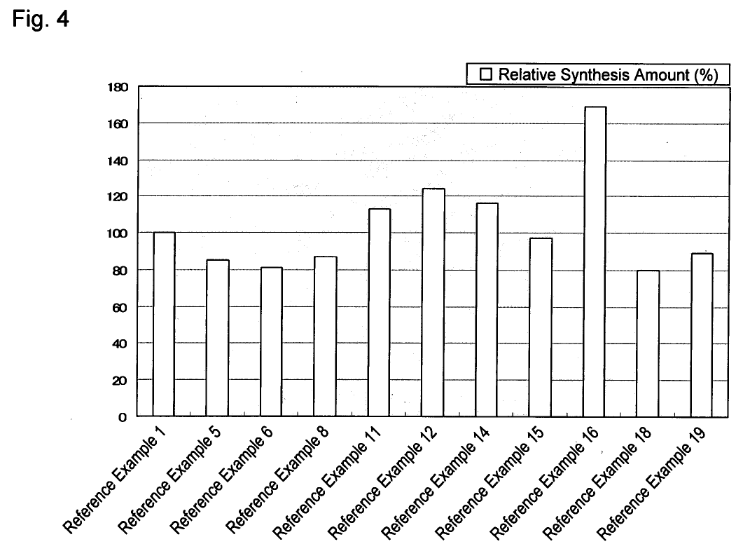

FIG. 4 is a graph showing a result of Experiment Example 5, in which a relative synthesis amount to mRAN transcribed from the vector pTNT-Luc produced in Reference Example 1 (100%) is represented on the vertical axis. Since the vector pTNT-Luc produced in Reference Example 1 has a DNA fragment (5′β-globin leader sequence) that suitably promotes a translation reaction in an extract solution derived from mammalians, in this context, a DNA fragment that exhibits 80% or more of synthesis amount relative to Reference Example 1 is referred to as a DNA fragment that promotes a translation reaction.

### Reference Example 24

### Production of Expression Vector (pTD1 Vector)

A primer having a base sequence represented by SEQ ID No. 17 of the sequence listing (T7 pMn-Eco) and an antisense strand thereof were synthesized by a DNA synthesizer, and their 5′ terminals were phosphorylated by T4 Polynucleotide Kinase. Following this reaction, the sense strand and the antisense strand were mixed and heated at 95° C. for 5 minutes. The mixture was allowed to cool to room temperature to make the sense strand and the antisense strand anneal. The product was then purified by ethanol precipitation and dissolved in water. After removing excess ATP by using Sigma Spin Post Reaction Purification Columns, purification by ethanol precipitation was conducted again. The resultant double-stranded DNA fragment was digested with EcoRI (manufactured by TOYOBO Co., Ltd.), to give an insert. Separately from this, pUC19 was digested with EheI (manufactured by TOYOBO Co., Ltd.) and EcoRI, and separation by electrophoresis was conducted, and then a DNA fragment of about 2.5 kb was purified by using Gen Elute Gel Purification Kit. The insert was ligated with the pUC19-derived vector and E. coli DH5α was transformed. After preparing a plasmid from the resultant E. coli cell, base sequence analysis using M13 Reverse primer represented by SEQ ID No. 18 of the sequence listing was conducted. The obtained plasmid DNA was named “pUM”.

Using 0.5 μg of BD BaculoGold Linearized Baculovirus DNA (manufactured by BD Biosciences) as a template, as well as a primer having a base sequence represented by SEQ ID No. 19 of the sequence listing (Phd3 Fw), primer having a base sequence represented by SEQ ID No. 20 of the sequence listing (Phd3 Rv-Hind), and KOD plus (manufactured by TOYOBO Co., Ltd.), after denaturing the template at 96° C. for 2 minutes, 30 cycles of PCR (each cycle including 96° C. 15 seconds, 50° C. 30 seconds, 68° C. 30 seconds) was conducted, thereby amplifying 3′UTR of polyhedrin gene of AcNPV (Autographa californica nuclear polyhedrosis virus). 5′ terminal of DNA fragment was phosphorylated by using T4 Polynucleotide Kinase. Following purification by ethanol precipitation, the reaction mixture was digested with HindIII (manufactured by TOYOBO Co., Ltd.), to give an insert. Separately from this, pUM was digested with HincII (manufactured by TOYOBO Co., Ltd.) and HindIII. Following separation by electrophoresis, a DNA fragment of about 2.7 kb was purified by using Gen Elute Gel Purification Kit. The insert was ligated with the pUM-derived vector and E. coli DH5α was transformed. After preparing a plasmid from the resultant E. coli cell, base sequence analysis using M13 Reverse primer represented by SEQ ID No. 18 of the sequence listing was conducted. The obtained plasmid DNA was named “pTM”.

A sense strand having a base sequence represented by SEQ ID No. 21 of the sequence listing (A25T7t) and an antisense strand of primer were synthesized by a DNA synthesizer, and their 5′ terminals were phosphorylated by T4 Polynucleotide Kinase. Following this reaction, the sense strand and the antisense strand were mixed and heated at 95° C. for 5 minutes. The mixture was allowed to cool to room temperature to make the sense strand and the antisense strand anneal. The product was then purified by ethanol precipitation and dissolved in water. After removing excess ATP by using Sigma Spin Post Reaction Purification Columns, purification by ethanol precipitation was conducted again, to give an insert. Separately from this, using 5 ng of pTM as a template, as well as a primer having a base sequence represented by SEQ ID No. 22 of the sequence listing (Not Fw), a primer having a base sequence represented by SEQ ID No. 23 of the sequence listing (Phd3 Rv), and KOD plus (manufactured by TOYOBO Co., Ltd.), after denaturing the template at 96° C. for 2 minutes, 30 cycles of PCR (each cycle including 96° C. 15 seconds, 50° C. 30 seconds, 68° C. 3 minutes) was conducted. After separating the PCR product by electrophoresis, a DNA fragment of about 3.0 kb was purified by using Gen Elute Gel Purification Kit. The purified PCR product and the insert were subjected to ligation, and E. coli DH5α was transformed. After preparing a plasmid from the resultant E. coli cell, base sequence analysis using M13 Reverse primer represented by SEQ ID No. 18 of the sequence listing was conducted. The plasmid DNA in which poly-A tail was inserted downstream side of AcNPV polyhedrin 3′UTR sequence in forward direction was named “pTMA”.

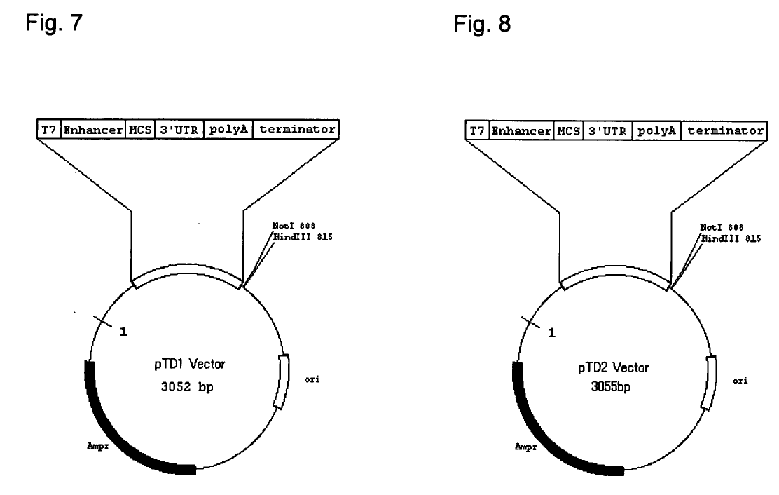

Using 5 ng of pTMA as a template, as well as a primer having a base sequence represented by SEQ ID No. 22 of the sequence listing (Not Fw), a primer having a base sequence represented by SEQ ID No. 24 of the sequence listing (Not Rv), and KOD plus (manufactured by TOYOBO Co., Ltd.), after denaturing the template at 96° C. for 2 minutes, 30 cycles of PCR (each cycle including 96° C. 15 seconds, 50° C. 30 seconds, 68° C. 3 minutes) was conducted. 5′ terminal of the PCR product was phosphorylated by using T4 Polynucleotide Kinase. After separating the reaction mixture by electrophoresis, a DNA fragment of about 3.0 kb was purified by using Gen Elute Gel Purification Kit. The purified PCR product was ligated, and E. coli DH5α was transformed. After preparing a plasmid from the resultant E. coli, base sequence analysis using M13 Reverse primer represented by SEQ ID No. 18 of the sequence listing was conducted. The obtained plasmid DNA was named “pTD1 Vector”. The produced pTD1 Vector has a base sequence represented by SEQ ID No. 29 of the sequence listing. FIG. 7 shows a map of the generated pTD1 Vector. In this map, “T7” means T7 promoter, “Enhancer” means 5′UTR of polyhedrin gene derived from MnNPV (forward direction), “MCS” means multi-cloning site, “3“UTR” means 3′UTR of polyhedrin gene derived from AcNPV, “poly-A” means poly-A tail, and “terminator” means T7 terminator sequence.

### Reference Example 25

### Production of Expression Vector (pTD2 Vector)

A primer having a base sequence represented by SEQ ID No. 25 of the sequence listing (T7pEo-Eco) and an antisense strand thereof were synthesized by a DNA synthesizer, and their 5′ terminals were phosphorylated by T4 Polynucleotide Kinase. Following this reaction” the sense strand and the antisense strand were mixed and heated at 95° C. for 5 minutes. The mixture was allowed to cool to room temperature to make the sense strand and the antisense strand anneal. The product was then purified by ethanol precipitation and dissolved in water. After removing excess ATP by using Sigma Spin Post Reaction Purification Columns, purification by ethanol precipitation was conducted again. The resultant double-stranded DNA fragment was digested with EcoRI (manufactured by TOYOBO Co., Ltd.), to give an insert. Separately from this, pUC19 was digested with EheI (manufactured by TOYOBO Co., Ltd.) and EcoRI and following separation by electrophoresis, a DNA fragment of about 2.5 kb was purified by using Gen Elute Gel Purification Kit. The pUC19-derived vector and the insert were subjected to ligation, and E. coli DH5α was transformed. After preparing a plasmid from the resultant E. coli, base sequence analysis using M13 Reverse primer represented by SEQ ID No. 18 of the sequence listing was conducted. The obtained plasmid DNA was named “pUE”.

Using 0.5 μg of BD BaculoGold Linearized Baculovirus DNA (manufactured by BD Biosciences) as a template, as well as primer having a base sequence represented by SEQ ID No. 19 of the sequence listing (Phd3 Fw), primer having a base sequence represented by SEQ ID No. 20 of the sequence listing (Phd3 Rv-Hind), and KOD plus (manufactured by TOYOBO Co., Ltd.), after denaturing the template at 96° C. for 2 minutes, 30 cycles of PCR (each cycle including 96° C. 15 seconds, 50° C. 30 seconds, 68° C. 30 seconds) was conducted, thereby amplifying 3′UTR of polyhedrin gene of AcNPV (Autographa californica nuclear polyhedrosis virus). 5′ terminal of DNA fragment was phosphorylated by using T4 Polynucleotide Kinase. Following purification by ethanol precipitation, the reaction mixture was digested with HindIII (manufactured by TOYOBO Co., Ltd.), to give an insert. Separately from this, pUE was digested with HincII (manufactured by TOYOBO Co., Ltd.) and HindIII. Following separation by electrophoresis, a DNA fragment of about 2.7 kb was purified by using Gen Elute Gel Purification Kit. The pUE-derived vector and the insert were subjected to ligation, and E. coli DH5α was transformed. After preparing a plasmid from the resultant E. coli, base sequence analysis using M13 Reverse primer represented by SEQ ID No. 18 of the sequence listing was conducted. The obtained plasmid DNA was named “pTE”.

A sense strand having a base sequence represented by SEQ ID No. 21 of the sequence listing (A25T7t) and an antisense strand of primer were synthesized by a DNA synthesizer, and their 5′ terminals were phosphorylated by T4 Polynucleotide Kinase. Following this reaction, the sense strand and the antisense strand were mixed and heated at 95° C. for 5 minutes. The mixture was allowed to cool to room temperature to make the sense strand and the antisense strand anneal. The product was then purified by ethanol precipitation and dissolved in water. After removing excess ATP by using Sigma Spin Post Reaction Purification Columns, purification by ethanol precipitation was conducted again, to give an insert. Separately from this, using 5 ng of pTE as a template, as well as a primer having a base sequence represented by SEQ ID No. 22 of the sequence listing (Not Fw), a primer having a base sequence represented by SEQ ID No. 23 of the sequence listing (Phd3 Rv), and KOD plus (manufactured by TOYOBO Co., Ltd.), after denaturing the template at 96° C. for 2 minutes, 30 cycles of PCR (each cycle including 96° C. 15 seconds, 50° C. 30 seconds, 68° C. 3 minutes) was conducted. After separating the PCR product by electrophoresis, a DNA fragment of about 3.0 kb was purified by using Gen Elute Gel Purification Kit. The purified PCR product and the insert were subjected to ligation, and E. coli DH5α was transformed. After preparing a plasmid from the resultant E. coli, base sequence analysis using M13 Reverse primer represented by SEQ ID No. 18 of the sequence listing was conducted. The plasmid DNA in which poly-A tail was inserted downstream side of AcNPV polyhedrin 3′UTR sequence in forward direction was named “pTEA”.

Using 5 ng of pTEA as a template, as well as a primer having a base sequence represented by SEQ ID No. 22 of the sequence listing (Not Fw), a primer having a base sequence represented by SEQ ID No. 24 of the sequence listing (Not Rv), and KOD plus (manufactured by TOYOBO Co., Ltd.), after denaturing the template at 96° C. for 2 minutes, 30 cycles of PCR (each cycle including 96° C. 15 seconds, 50° C. 30 seconds, 68° C. 3 minutes) was conducted. 5′ terminal of the PCR product was phosphorylated by using T4 Polynucleotide Kinase. After separating the reaction mixture by electrophoresis, a DNA fragment of about 3.0 kb was purified by using Gen Elute Gel Purification Kit. The purified PCR product was ligated, and E. coli DH5α was transformed. After preparing a plasmid from the resultant E. coli, base sequence analysis using M13 Reverse primer represented by SEQ ID No. 18 of the sequence listing was conducted. The obtained plasmid DNA was named “pTD2 Vector”.

The produced pTD2 Vector has a base sequence represented by SEQ ID No. 30 of the sequence listing. FIG. 8 shows a map of the generated pTD1 Vector. In this map, “T7” means T7 promoter, “Enhancer” means 5′UTR of polyhedrin gene derived from EoNPV (reverse direction), “MCS” means multi-cloning site, “3′UTR” means 3′UTR of polyhedrin gene derived from AcNPV, “poly-A” means poly-A tail, and “terminator” means T7 terminator sequence.

### Example 1

### Gene Cloning Using pTD1 Vector and Cell-Free Protein Synthesis System Using the Same

(1) Production of Template DNA (Vector pTD1-Luc)

Using 5 ng of pGEM-Luc Vector (manufactured by Promega Corporation) having a structural gene encoding luciferase as a template, as well as a primer having a base sequence represented by SEQ ID No. 16 of the sequence listing (Luc-ATG), a primer having a base sequence represented by SEQ ID No. 13 of the sequence listing (Luc T7-R4-Kpn), and KOD plus (manufactured by TOYOBO Co., Ltd.), after denaturing the template at 96° C. for 2 minutes, 30 cycles of PCR (each cycle including 96° C. 15 seconds, 50° C. 30 seconds, 68° C. 120 seconds) was conducted, thereby amplifying the open reading frame (ORF). After phosphorylating 5′ terminal of the PCR product by using T4 Polynucleotide Kinase, the PCR product was purified by ethanol precipitation. After digesting with KpnI, the resultant DNA fragment was subjected to electrophoresis, and a DNA fragment of about 1.6 kb was purified by using Gen Elute Gel Purification Kit, to give an insert. Separately from this, using 5 ng of pTD1 Vector as a template, as well as a primer having a base sequence represented by SEQ ID No. 26 of the sequence listing (Eco-Kpn), a primer having a base sequence represented by SEQ ID No. 27 of the sequence listing (Mn29 Rv), and KOD plus (manufactured by TOYOBO Co., Ltd.), after denaturing the template at 96° C. for 2 minutes, 30 cycles of PCR (each cycle including 96° C. 15 seconds, 50° C. 30 seconds, 68° C. 3 minutes) was conducted. After purification by ethanol precipitation, the PCR product was digested with KpnI. After separation by electrophoresis, a DNA fragment of about 3.0 kb was purified using Gen Elute Gel Purification Kit. The pTD1 Vector-derived DNA fragment and the insert were subjected to ligation, and E. coli DH5α was transformed. After preparing a plasmid from the resultant E. coli, base sequence analysis was conducted using a primer having a base sequence represented by SEQ ID No. 14 of the sequence listing (T7 promoter) and M13 Reverse primer represented by SEQ ID No. 18 of the sequence listing. The obtained plasmid DNA was named “pTD1-Luc”.

(2) In Vitro Transcription Reaction and Purification of mRNA

In vitro transcription reaction and purification of mRNA were conducted in the same manner as described in Experiment Example 1.

(3) Translation Reaction

For translation reaction, the method described in Experiment Example 3 was followed using the extract solution of insect culture cell (Sf21) produced in Reference Example 21. Synthesized luciferase was quantified in accordance with the method described in Experiment Example 3.

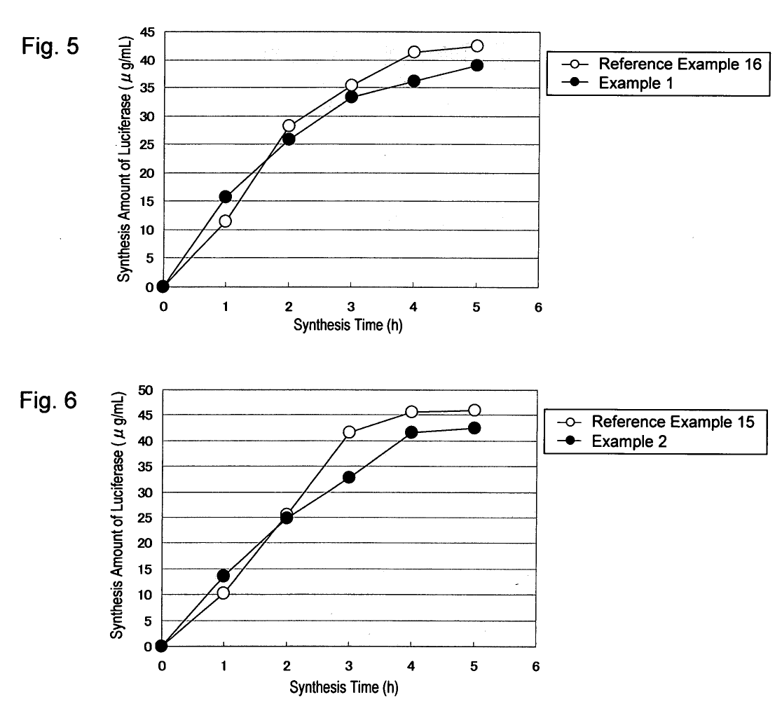

FIG. 5 is a graph showing a result of Example 1 in which the horizontal axis represents synthesis time (hour) and the vertical axis represents synthesis amount of luciferase (μg/mL) For comparison, also a result of synthesis using mRNA transcribed from pMphd-FF-Luc produced in Reference Example 16 is shown. As shown in FIG. 5, it was demonstrated that the protein expression vector produced in Reference Example 24 by using a DNA fragment having a base sequence represented by SEQ ID No. 9 of the sequence listing as a DNA fragment promoting translation reaction exhibited the same degree of synthesis amount as the case where pMphd-FF-Luc produced in Reference Example 16 was used as a template DNA.

### Example 2

### Gene Cloning Using pTD2 Vector and Cell-Free Protein Synthesis System Using the Same

(1) Production of Template DNA (Vector pTD2-Luc)

Using 5 ng of pGEM-Luc Vector (manufactured by Promega Corporation) having a structural gene encoding luciferase as a template, as well as a primer having a base sequence represented by SEQ ID No. 16 of the sequence listing (Luc-ATG), a primer having a base sequence represented by SEQ ID No. 13 of the sequence listing (Luc T7-R4-Kpn), and KOD plus (manufactured by TOYOBO Co., Ltd.), after denaturing the template at 96° C. for 2 minutes, 30 cycles of PCR (each cycle including 96° C. 15 seconds, 50° C. 30 seconds, 68° C. 120 seconds) was conducted, thereby amplifying the open reading frame (ORF). After phosphorylating 5′ terminal of the PCR product by using T4 Polynucleotide Kinase, the PCR product was purified by ethanol precipitation. The DNA fragment was digested with KpnI, and then subjected to electrophoresis, and a DNA fragment of about 1.6 kb was purified by using Gen Elute Gel Purification Kit, to give an insert. Separately from this, using 5 ng of pTD2 Vector as a template, as well as a primer having a base sequence represented by SEQ ID No. 26 of the sequence listing (Eco-Kpn), a primer having a base sequence represented by SEQ ID No. 28 of the sequence listing (Eo21 Fw), and KOD plus (manufactured by TOYOBO Co., Ltd.), after denaturing the template at 96° C. for 2 minutes, 30 cycles of PCR (each cycle including 96° C. 15 seconds, 50° C. 30 seconds, 68° C. 3 minutes) was conducted. After purification by ethanol precipitation, the PCR product was digested with KpnI. After separation by electrophoresis, a DNA fragment of about 3.0 kb was purified using Gen Elute Gel Purification Kit. The pTD2 Vector-derived DNA fragment and the insert were subjected to ligation, and E. coli DH5α was transformed. After preparing a plasmid from the resultant E. coli, base sequence analysis was conducted using a primer having a base sequence represented by SEQ ID No. 14 of the sequence listing (T7 promoter) and M13 Reverse primer represented by SEQ ID No. 18 of the sequence listing. The obtained plasmid DNA was named “pTD2-Luc”.

(2) In Vitro Transcription Reaction and Purification of mRNA

In vitro transcription reaction and purification of mRNA were conducted in the same manner as described in Experiment Example 1.

(3) Translation Reaction

For translation reaction, the method described in Experiment Example 2 was followed using the extract solution of insect culture cell (High Five) produced in Reference Example 20. Synthesized luciferase was quantified in accordance with the method described in Experiment Example 2.

FIG. 6 is a graph showing a result of Example 2 in which the horizontal axis represents synthesis time (hour) and the vertical axis represents synthesis amount of luciferase (μg/mL) For comparison, also a result of synthesis using mRNA transcribed from pEophd-R-Luc produced in Reference Example 15 is shown. As shown in FIG. 6, it was demonstrated that the protein expression vector produced in Reference Example 25 by using a DNA fragment having a base sequence represented by SEQ ID No. 8 of the sequence listing as a DNA fragment promoting translation reaction exhibited the same degree of synthesis amount as the case where pEophd-R-Luc produced in Reference Example 15 was used as a template DNA.

### Reference Example 26

### Production of Template DNA (Vector pEU3-N-2-Luc)

Using 5 ng of pGEM-Luc Vector (manufactured by Promega Corporation) having a structural gene encoding luciferase as a template, as well as a primer having a base sequence represented by SEQ ID No. 16 of the sequence listing (Luc-ATG), a primer having a base sequence represented by SEQ ID No. 13 of the sequence listing (Luc T7-R4-Kpn), and KOD plus (manufactured by TOYOBO Co., Ltd.), after denaturing the template at 96° C. for 2 minutes, 30 cycles of PCR (each cycle including 96° C. 15 seconds, 50° C. 30 seconds, 68° C. 120 seconds) was conducted, thereby amplifying the open reading frame (ORF). After phosphorylating 5′ terminal of the PCR product by using T4 Polynucleotide Kinase, the PCR product was purified by ethanol precipitation. The DNA fragment was digested with KpnI, and then subjected to electrophoresis, and a DNA fragment of about 1.6 kb was purified by using Gen Elute Gel Purification Kit, to give an insert. Separately from this, pEU3-N2 Vector (expression vector for wheat germ extract solution having Ω sequence derived from tobacco mosaic virus as a translation promoting sequence, manufactured by TOYOBO Co., Ltd.) was digested with EcoRV and KpnI, to which the insert produced above was ligated, and then E. coli DH5α was transformed. After preparing a plasmid from the resultant E. coli cell, base sequence analysis was conducted using a primer having a base sequence represented by SEQ ID No. 14 of the sequence listing (T7 promoter) and M13 Reverse primer. The obtained plasmid DNA was named “pEU3-N-2-Luc”. This plasmid for protein expression is expected to be suitably expressed in cell-free protein synthesis system using a wheat germ extract solution.

### Example 3

### Cell-Free Protein Synthesis System with Wheat Germ Extract Solution Using pTD1 Vector

(1) Template DNA

As a template DNA, pTD1-Luc produced in Example 1 was used.

(2) In Vitro Transcription Reaction and Purification of mRNA

In vitro transcription reaction and purification of mRNA was conducted in the same manner as described in Experiment Example 1.

(3) Translation Reaction

As to translation reaction, cell-free protein synthesis system was conducted using the mRNA produced in the above (2) as a template and a wheat germ extraction solution. As the wheat germ extraction solution, PROTEIOSTM ver. 2 (manufactured by TOYOBO Co., Ltd.) was used. mRNA was added so that its final concentration was 240 μg/mL, and protein synthesis reaction was conducted in a reaction scale of 50 μL (batch reaction) according to an instruction manual. The synthesized luciferase was quantified in accordance with the method described in Experiment Example 2.

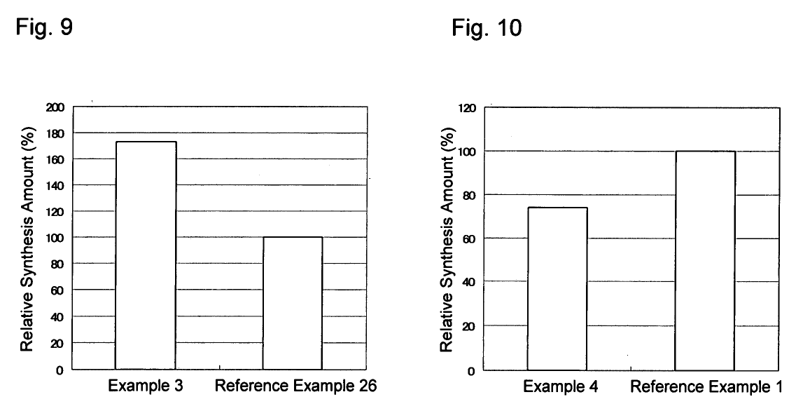

FIG. 9 shows a result in which a relative synthesis amount (%) of luciferase is represented along the vertical axis. For reference, a result of cell-free protein synthesis system using a wheat germ extract solution conducted by using pEU3-N-2-Luc produced in Reference Example 26 as a template DNA (Reference Example 26) is shown. As shown in FIG. 9, the synthesis amount by pTD1-Luc produced in Example 1 was about 170% of that synthesized by pEU3-N-2-Luc, which revealed that pTD1 Vector was suitably used as the expression vector capable of promoting translation reaction even in cell-free protein synthesis system using a wheat germ extract solution.

### Example 4

### Cell-Free Protein Synthesis System with Rabbit Reticulocyte Extract Solution Using pTD1 Vector

(1) Template DNA

As a template DNA, pTD1-Luc produced in Example 1 was used.

(2) In Vitro Transcription Reaction and Purification of mRNA

In vitro transcription reaction and purification of mRNA was conducted in the same manner as described in Experiment Example 1.

(3) Translation Reaction

As to translation reaction, cell-free protein synthesis system was conducted using the mRNA produced in the above (2) as a template and a rabbit reticulocyte extract solution. As the rabbit reticulocyte extract solution, Rabbit Reticulocyte Lysate, Nuclease Treated (manufactured by Promega Corporation) was used. mRNA was added so that its final concentration was 40 μg/mL, and protein synthesis reaction was conducted in a reaction scale of 50 μL according to an instruction manual. The synthesized luciferase was quantified in accordance with the method described in Experiment Example 2.

The result is shown in FIG. 10. A relative synthesis amount (%) of luciferase is represented along the vertical axis. For reference, a result of cell-free protein synthesis system using a rabbit reticulocyte extract solution conducted by using pTNT-Luc produced in Reference Example 1 as a template DNA (Reference Example 1) is shown. As shown in FIG. 10, in cell-free protein synthesis system using a rabbit reticulocyte extract solution, pTD1-Luc exhibited a similar degree of synthesis amount as pTNT-LUC, which revealed that pTD1 Vector was suitably used as the expression vector capable of promoting translation reaction even in cell-free protein synthesis system using a rabbit reticulocyte extract solution.

In Reference Examples 24 and 25, the protein expression vectors were produced respectively using DNA fragments having base sequences represented by SEQ ID Nos. 9 and 8 of the sequence listing as DNA fragments having translation reaction activity, however, protein expression vectors using DNA fragments having base sequences represented by other SEQ ID Nos. may be readily produced.

It is also clear from Experiment Examples 2-5 and Examples 1-4 that synthesis amount of protein in cell-free protein synthesis system is improved by using such expression vectors.

The present invention can be carried out in various other modes. Therefore, the above-described Example is merely illustrative in all respects, and must not be construed as being restrictive. Further, the changes that fall within the equivalents of the claims are all within the scope of the present invention.

What is claimed is:

1. A DNA fragment of any of the following (a) to (l) used for promoting a translation reaction in a cell-free protein synthesis system:
(a) a DNA fragment having a base sequence represented by SEQ ID No. 1 of the sequence listing;
(b) a DNA fragment having a base sequence represented by SEQ ID No. 2 of the sequence listing;
(c) a DNA fragment having a base sequence represented by SEQ ID No. 3 of the sequence listing;
(d) a DNA fragment having a base sequence represented by SEQ ID No. 4 of the sequence listing;
(e) a DNA fragment having a base sequence represented by SEQ ID No. 5 of the sequence listing;
(f) a DNA fragment having a base sequence represented by SEQ ID No. 6 of the sequence listing;
(g) a DNA fragment having a base sequence represented by SEQ ID No. 7 of the sequence listing;
(h) a DNA fragment having a base sequence represented by SEQ ID No. 8 of the sequence listing;
(i) a DNA fragment having a base sequence represented by SEQ ID No. 9 of the sequence listing;
(j) a DNA fragment having a base sequence represented by SEQ ID No. 10 of the sequence listing;
(k) a DNA fragment having a base sequence represented by SEQ ID No. 11 of the sequence listing; and
(l) a DNA fragment having a base sequence in which one or several base(s) is/are deleted, substituted, inserted or added from/to a base sequence represented by any of SEQ ID Nos. 1-11 of the sequence listing, and having a translation reaction promoting activity.

2. An expression vector containing at least one DNA fragment selected from the group consisting of the following (a) to (l) having a translation reaction promoting activity:
(a) a DNA fragment having a base sequence represented by SEQ ID No. 1 of the sequence listing;
(b) a DNA fragment having a base sequence represented by SEQ ID No. 2 of the sequence listing;
(c) a DNA fragment having a base sequence represented by SEQ ID No. 3 of the sequence listing;
(d) a DNA fragment having a base sequence represented by SEQ ID No. 4 of the sequence listing;
(e) a DNA fragment having a base sequence represented by SEQ ID No. 5 of the sequence listing;
(f) a DNA fragment having a base sequence represented by SEQ ID No. 6 of the sequence listing;
(g) a DNA fragment having a base sequence represented by SEQ ID No. 7 of the sequence listing;
(h) a DNA fragment having a base sequence represented by SEQ ID No. 8 of the sequence listing;
(i) a DNA fragment having a base sequence represented by SEQ ID No. 9 of the sequence listing;
(j) a DNA fragment having a base sequence represented by SEQ ID No. 10 of the sequence listing; and
(k) a DNA fragment having a base sequence represented by SEQ ID No. 11 of the sequence listing; and
(l) a DNA fragment having a base sequence in which one or several base(s) is/are deleted, substituted, inserted or added from/to a base sequence represented by any of SEQ ID Nos. 1-11 of the sequence listing, and having a translation reaction promoting activity.

3. A template DNA for cell-free protein synthesis system having a structural gene encoding a protein and a DNA fragment incorporated upstream side of 5′ of the structural gene, wherein the DNA fragment is selected from the group consisting of the following (a) to (l) having a translation reaction promoting activity:
(a) a DNA fragment having a base sequence represented by SEQ ID No. 1 of the sequence listing;
(b) a DNA fragment having a base sequence represented by SEQ ID No. 2 of the sequence listing;
(c) a DNA fragment having a base sequence represented by SEQ ID No. 3 of the sequence listing;
(d) a DNA fragment having a base sequence represented by SEQ ID No. 4 of the sequence listing;
(e) a DNA fragment having a base sequence represented by SEQ ID No. 5 of the sequence listing;
(f) a DNA fragment having a base sequence represented by SEQ ID No. 6 of the sequence listing;
(g) a DNA fragment having a base sequence represented by SEQ ID No. 7 of the sequence listing;
(h) a DNA fragment having a base sequence represented by SEQ ID No. 8 of the sequence listing;
(i) a DNA fragment having a base sequence represented by SEQ ID No. 9 of the sequence listing;
(j) a DNA fragment having a base sequence represented by SEQ ID No. 10 of the sequence listing; and
(k) a DNA fragment having a base sequence represented by SEQ ID No. 11 of the sequence listing; and
(l) a DNA fragment having a base sequence in which one or several base(s) is/are deleted, substituted, inserted or added from/to a base sequence represented by any of SEQ ID Nos. 1-11 of the sequence listing, and having a translation reaction promoting activity.

4. A mRNA obtained by transcription from the template DNA according to claim 3 and used as a transcription template in cell-free protein synthesis system.

5. A reaction solution for cell-free protein synthesis system including the template DNA according to claim 3 or the mRNA obtained by transcription from the template DNA.

6. A method for cell-free protein synthesis system using the template DNA according to claim 3 or the mRNA obtained by transcription from the template DNA.

7. The method for cell-free protein synthesis system according to claim 6, using a reaction solution for cell-free protein synthesis system including an animal-derived extract.

8. The method for cell-free protein synthesis system according to claim 7, wherein the animal-derived extract is extracted from a silk worm tissue.

9. The method for cell-free protein synthesis system according to claim 7, wherein the animal-derived extract is extracted from an insect culture cell.

10. The method for cell-free protein synthesis system according to claim 9, wherein the insect culture cell is a cell derived from Trichoplusia ni egg cell and/or Spodoptera frugiperda ovary cell.

11. The method for cell-free protein synthesis system according to claim 7, wherein the animal-derived extract is extracted from a mammalian cell.

12. The method for cell-free protein synthesis system according to claim 11, wherein the mammalian cell is a rabbit reticulocyte.

13. The method for cell-free protein synthesis system according to claim 11, wherein the mammalian cell is a mammalian culture cell.

14. The method for cell-free protein synthesis system according to claim 13, wherein the mammalian culture cell is a Chinese hamster ovary cell.

15. The method for cell-free protein synthesis system according to claim 6, using a reaction solution for cell-free protein synthesis system including a wheat germ extract.

16. A kit for cell-free protein synthesis system including the expression vector according to claim 2.


SEQUENCE LISTING (30 sequences)

SEQ ID NO: 1 - DNA, 42 bp, Artificial Sequence
  5′UTR (Bombyx mori fibroin L-chain gene)
ctgtatagtatataccgattggtcacataacagaccactaaa

SEQ ID NO: 2 - DNA, 54 bp, Artificial Sequence
  5′UTR (Bombyx mori sericin gene)
atagtcgtcttatcatcgggtctctaaggatcaagcgatccaaagaccgccaac

SEQ ID NO: 3 - DNA, 49 bp, Artificial Sequence
  5′UTR (AcNPV polyhedrin gene)
agtattttactgttttcgtaacagttttgtaataaaaaaacctataaat

SEQ ID NO: 4 - DNA, 41 bp, Artificial Sequence
  5′UTR (BmCPV polyhedrin gene)
agtaaaagtcagtatcttaccggcataatacgtaaaggatc

SEQ ID NO: 5 - DNA, 34 bp, Artificial Sequence
  5′UTR (EsCPV polyhedrin gene)
agtttaaaatcctcagcggaattaaaacaccaag

SEQ ID NO: 6 - DNA, 42 bp, Artificial Sequence
  5′UTR (HcNPV polyhedrin gene)
gctgttattgtagcaattttgtaataaaaatatcctataact

SEQ ID NO: 7 - DNA, 49 bp, Artificial Sequence
  5′UTR (CrNPV polyhedrin gene)
agtaatttgctggttttgtagcaattttgtaatataatttcgtataact

SEQ ID NO: 8 - DNA, 49 bp, Artificial Sequence
  5′UTR (EoNPV polyhedrin gene)
agtattgtagtcctttcgtaattgtttgtgaaatctaaaatacaccgta

SEQ ID NO: 9 - DNA, 46 bp, Artificial Sequence
  5′UTR (MnNPV polyhedrin gene)
agtatttttattctttcgtaaaaaaattagaaaaataaaatataaa

SEQ ID NO: 10 - DNA, 40 bp, Artificial Sequence
  5′UTR (SfNPV polyhedrin gene)
agtaattttttcctttcgtaaaacattgtgaaaaaataaa

SEQ ID NO: 11 - DNA, 48 bp, Artificial Sequence
  5′UTR (WsNPV polyhedrin gene)
agaattttagagtaatcgttcgcgattgtgaaaaaaaggtttgccata

SEQ ID NO: 12 - DNA, 30 bp, Artificial Sequence
  Luc T7-F3-Kpn
ggggtaccatggaagacgccaaaaacataa

SEQ ID NO: 13 - DNA, 29 bp, Artificial Sequence
  Luc T7-R4-Kpn
ggggtaccttacaatttggactttccgcc

SEQ ID NO: 14 - DNA, 21 bp, Artificial Sequence
  T7 promoter
cttaatacgactcactatagg

SEQ ID NO: 15 - DNA, 21 bp, Artificial Sequence
  T7p Rv
cctatagtgagtcgtattaag

SEQ ID NO: 16 - DNA, 22 bp, Artificial Sequence
  Luc-ATG
atggaagacgccaaaaacataa

SEQ ID NO: 17 - DNA, 80 bp, Artificial Sequence
  T7pMn-Eco
cttaatacgactcactataggagtatttttattctttcgtaaaaaaattagaaaaataaaatataaagatatcgaattcg

SEQ ID NO: 18 - DNA, 19 bp, Artificial Sequence
  M13Reverse
ggaaacagctatgaccatg

SEQ ID NO: 19 - DNA, 35 bp, Artificial Sequence
  Phd3 Fw
gggcggctgtaaaacacgatacattgttattagta

SEQ ID NO: 20 - DNA, 35 bp, Artificial Sequence
  Phd3 Rv-Hind
cccaagcttgcggataatattttgaacgacgtcga

SEQ ID NO: 21 - DNA, 73 bp, Artificial Sequence
  A25T7t
aaaaaaaaaaaaaaaaaaaaaaaaactagcataaccccttggggcctctaaacgggtcttgaggggttttttg

SEQ ID NO: 22 - DNA, 22 bp, Artificial Sequence
  Not Fw
cgcaagcttggcgtaatcatgg

SEQ ID NO: 23 - DNA, 26 bp, Artificial Sequence
  Phd3 Rv
gcggataatattttgaacgacgtcga

SEQ ID NO: 24 - DNA, 24 bp, Artificial Sequence
  Not Rv
gccgcaaaaaacccctcaagaccc

SEQ ID NO: 25 - DNA, 83 bp, Artificial Sequence
  T7pEo-Eco
cttaatacgactcactataggtacggtgtattttagatttcacaaacaattacgaaaggactacaatactgatatcgaattcg

SEQ ID NO: 26 - DNA, 23 bp, Artificial Sequence
  Eco-Kpn
gatatcgaattcgagctcggtac

SEQ ID NO: 27 - DNA, 29 bp, Artificial Sequence
  Mn29 Rv
tttatattttatttttctaatttttttac

SEQ ID NO: 28 - DNA, 21 bp, Artificial Sequence
  Eo21 Fw
agtattgtagtcctttcgtaa

SEQ ID NO: 29 - DNA, 3052 bp, Artificial Sequence
  pTD1 Vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggccttaatacgactcactataggagtatttttattctttcgtaaaaaaattagaaaaataaaatataaagatatcgaattcgagctcggtacccggggatcctctagagtcgggcggctgtaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatccgcaaaaaaaaaaaaaaaaaaaaaactagcataaccccttggggcctctaaacgggtcttgaggggttttttgcggccgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 30 - DNA, 3055 bp, Artificial Sequence
  pTD2 Vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggccttaatacgactcactataggtacggtgtattttagatttcacaaacaattacgaaaggactacaatactgatatcgaattcgagctcggtacccggggatcctctagagtcgggcggctgtaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatccgcaaaaaaaaaaaaaaaaaaaaaactagcataaccccttggggcctctaaacgggtcttgaggggttttttgcggccgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc